(19) United States
(12) Patent Application Publication
(10) Pub. No.: US 2024/0216271 A1
(43) Pub. Date: Jul. 4, 2024

(54) HYDROLYTICALLY DEGRADABLE HYDROGELS AND USES THEREOF

(71) Applicant: GEORGIA TECH RESEARCH CORPORATION, Atlanta, GA (US)

(72) Inventors: Andres J. GARCIA, Atlanta, GA (US); Maria M. CORONEL, Atlanta, GA (US)

(21) Appl. No.: 18/563,543

(22) PCT Filed: May 26, 2022
(86) PCT No.: PCT/US2022/031095

§ 371 Date: Nov. 22, 2023

Related U.S. Application Data
(60) Provisional application No. 63/193,211, filed on May 26, 2021.

(51) Int. Cl. A61K 9/06; A61K 47/34

(52) U.S. Cl. CPC A61K 9/06; A61K 47/34

(57) ABSTRACT

The present disclosure provides hydrogels, more particularly hydrolytically degradable hydrogels containing cleavable ester moieties and their use in such applications as tissue engineering and therapeutic delivery.

### CROSS-REFERENCE TO RELATED APPLICATIONS

This application claims the benefit of priority to U.S. Provisional Application No. 63/193,211, filed May 26, 2021, the disclosure of which is incorporated herein by reference in its entirety.

### TECHNICAL FIELD

This disclosure relates to hydrogels, and more particularly to hydrolytically degradable hydrogels which may find use in such applications as tissue engineering and therapeutic delivery.

### BACKGROUND

Advancements in therapeutic delivery mechanisms for the release of cargo molecules and cells has been propelled forward by research in cell-compatible biomaterials and encapsulation methods. Synthetic hydrogels, fabricated networks of crosslinked polymer structures, have been utilized to encapsulate bioactive materials, like growth factors and cellular products, generating 3D structures that can support and modulate cell behavior, with limited effect on viability and bioactive cargo efficacy (Guan, X., Avci-Adali, M., Alarçin, E., Cheng, H., Kashaf, S. S., Li, Y., Chawla, A., Jang, H. L., & Khademhosseini, A. (2017). Development of hydrogels for regenerative engineering. Biotechnology Journal, 12(5), 1600394). The tunability of mechanical properties, such as stiffness and matrix integrity, of these hydrogel systems, provides flexibility for use in a variety of microenvironments (Saxena, S., Hansen, C. E., & Lyon, L. A. (2014). Microgel Mechanics in Biomaterial Design. Accounts of Chemical Research, 47(8), 2426-2434 and Guan, X., Avci-Adali, M., Alarçin, E., Cheng, H., Kashaf, S. S., Li, Y., Chawla, A., Jang, H. L., & Khademhosseini, A. (2017). Development of hydrogels for regenerative engineering. Biotechnology Journal, 12(5), 1600394). Furthermore, the ability to implement degradable chemistries for fabrication constitutes a major advantage for noninvasive regenerative medicine applications, as post-degradation, the breakdown components can be excreted out of the body through renal filtration (Saxena, S., Hansen, C. E., & Lyon, L. A. (2014). Microgel Mechanics in Biomaterial Design. Accounts of Chemical Research, 47(8), 2426-2434 and Ulbrich, K. (1995). Synthesis of novel hydrolytically degradable hydrogels for controlled drug release. Journal of Controlled Release, 34(2), 155-165).

Hydrogel microparticles (microgels), either in suspension or as building blocks for granular bulk hydrogels, have emerged in recent years as an attractive platform in biomedical applications because of their highly tunable mechanical properties, injectability, and a high degree of tissue integration (Daly, A. C.; Riley, L.; Segura, T.; Burdick, J. A. Hydrogel Microparticles for Biomedical Applications. Nat Rev Mater 2020, 5 (1), 20-43). One of the design parameters that is directly coupled to microgel physical properties (e.g. stiffness, mesh size, etc.) is the degradation rate. Mechanisms for degradable crosslinking of polymers can be broadly categorized into enzymatic, photodegradable, hydrolytic, or a combination of these conferring varying degrees of control over degradation rates (Koh, J.; Griffin, D. R.; Archang, M. M.; Feng, A.-C.; Horn, T.; Margolis, M.; Zalazar, D.; Segura, T.; Scumpia, P. O.; Di Carlo, D. Enhanced In Vivo Delivery of Stem Cells Using Microporous Annealed Particle Scaffolds. Small 2019, 15 (39), 1903147, Griffin, D. R.; Weaver, W. M.; Scumpia, P. O.; Di Carlo, D.; Segura, T. Accelerated Wound Healing by Injectable Microporous Gel Scaffolds Assembled from Annealed Building Blocks. Nature Mater 2015, 14 (7), 737-744, Muir, V. G.; Qazi, T. H.; Shan, J.; Groll, J.; Burdick, J. A. Influence of Microgel Fabrication Technique on Granular Hydrogel Properties. ACS Biomater. Sci. Eng. 2021, 7 (9), 4269-4281, Foster, G. A.; Headen, D. M.; González-García, C.; Salmerón-Sánchez, M.; Shirwan, H.; García, A. J. Protease-Degradable Microgels for Protein Delivery for Vascularization. Biomaterials 2017, 113, 170-175, Photodegradable Hydrogels for Dynamic Tuning of Physical and Chemical Properties https://www.science.org/doi/10.1126/science.1169494 (accessed 2021-10-25), and Carleton, M. M.; Sefton, M. V. Injectable and Degradable Methacrylic Acid Hydrogel Alters Macrophage Response in Skeletal Muscle. Biomaterials 2019, 223, 119477). The majority of these methods are dependent on stimuli that are not easily controlled, spatially nor temporally (Jo, Y. S.; Gantz, J.; Hubbell, J. A.; Lutolf, M. P. Tailoring Hydrogel Degradation and Drug Release via Neighboring Amino Acid Controlled Ester Hydrolysis. Soft Matter 2009, 5 (2), 440-446).

Various polymer networks and crosslinking structures can be used to modulate the degradability and diffusivity of hydrogels allowing for an intricate control on the degradation rate and release profile of this platform (Jain, E., Hill, L., Canning, E., Sell, S. A., & Zustiak, S. P. (2017)). Control of gelation, degradation and physical properties of polyethylene glycol hydrogels through the chemical and physical identity of the crosslinker. Journal of Materials Chemistry B, 5(14), 2679-2691). Several mechanisms for degradable crosslinking of hydrogels have been explored including enzymatic, photodegradable, ester-based hydrolysis or a combination of these with varying degrees of control on degradation rates (Sung, B., Kim, C., & Kim, M.-H. (2015). Biodegradable colloidal microgels with tunable thermosensitive volume phase transitions for controllable drug delivery. Journal of Colloid and Interface Science, 450, 26-33, Stukel, J., Thompson, S., Simon, L., & Willits, R. (2015). Polyethlyene glycol microgels to deliver bioactive nerve growth factor: Microgels to Deliver Bioactive NGF. Journal of Biomedical Materials Research Part A, 103(2), 604-613, and Kloxin, A. M., Kasko, A. M., Salinas, C. N., & Anseth, K. S. (2009). Photodegradable Hydrogels for Dynamic Tuning of Physical and Chemical Properties. Science, 324(5923), 59-63). An increasingly prevalent degradation technique has focused on sequence-specific enzymatic degradation, whereby release is dependent on the proteolytic gel degradation carried out by cells and endogenous enzymatic release (Kroger, S. M., Hill, L., Jain, E., Stock, A., Bracher, P. J., He, F., & Zustiak, S. P. (2020). Design of Hydrolytically Degradable Polyethylene Glycol Crosslinkers for Facile Control of Hydrogel Degradation. Macromolecular Bioscience, 20(10), 2000085 and Lueckgen, A., Garske, D. S., Ellinghaus, A., Mooney, D. J., Duda, G. N., & Cipitria, A. (2019). Enzymatically-degradable alginate hydrogels promote cell spreading and in vivo tissue infiltration. Biomaterials, 217, 119294). By altering the amino acid sequence of the peptide cross-linker, degradation can be tuned, to the type of encapsulated cells, as well as the expected transplant milieu. Although this method has shown promise, degradation is dependent on external stimuli that are not easily controllable, spatially nor temporally (Jo, Y. S., Gantz, J., Hubbell, J. A., & Lutolf, M. P. (2009). Tailoring hydrogel degradation and drug release via neighboring amino acid-controlled ester hydrolysis. Soft Matter, 5(2), 440-446). Additionally, the high volume of enzymatically cleavable peptide linkers that are used during fabrication makes this a costly method to scale up into clinically relevant sizes and has the potential to be immunogenic as degradable peptide sequences can be recognized by the host immune system (Griffin, D. R., Archang, M. M., Kuan, C. H., Weaver, W. M., Weinstein, J. S., Feng, A. C., Ruccia, A., Sideris, E., Ragkousis, V., Koh, J., Plikus, M. V., Di Carlo, D., Segura, T., & Scumpia, P. O. (2020). Activating an adaptive immune response from a hydrogel scaffold imparts regenerative wound healing [Preprint]. Bioengineering). Another chemistry that has been heavily developed is the photolytic cleavage of hydrogel linkers. This method of degradation relies on an external source of light through the use of a photo-cleavable compound as the crosslinker during fabrication (Ji, H., Xi, K., Zhang, Q., & Jia, X. (2017). Photodegradable hydrogels for external manipulation of cellular microenvironments with real-time monitoring. RSC Advances, 7(39), 24331-24337, Villiou, M., Paez, J. I., & del Campo, A. (2020). Photodegradable Hydrogels for Cell Encapsulation and Tissue Adhesion. ACS Applied Materials & Interfaces, 12(34), 37862-37872, and Kloxin, A. M., Kasko, A. M., Salinas, C. N., & Anseth, K. S. (2009). Photodegradable Hydrogels for Dynamic Tuning of Physical and Chemical Properties. Science, 324(5923), 59-63). Photodegradable hydrogels have been used in different applications, such as tissue adhesion, where cell-containing hydrogels are depolymerized via a controlled light source allowing for the immediate release of cells and debonding from tissues (Villiou, M., Paez, J. I., & del Campo, A. (2020). Photodegradable Hydrogels for Cell Encapsulation and Tissue Adhesion. ACS Applied Materials & Interfaces, 12(34), 37862-37872). This favorable for applications such as wound dressings and controlled cell therapy treatments where the release rate can be controlled (Villiou, M., Paez, J. I., & del Campo, A. (2020). Photodegradable Hydrogels for Cell Encapsulation and Tissue Adhesion. ACS Applied Materials & Interfaces, 12(34), 37862-37872). However, given the need for patient compliance and tissue depth limitations photodegradation is most likely not the best option for long-term cargo release.

There is a clear need for hydrogels capable of controlled degradation in vivo which may be useful in tissue engineering and therapeutic delivery applications. This disclosure addresses this, as well as other, needs.

### SUMMARY

The present disclosure provides hydrogels which are hydrolytically degradable and which are capable of controlled degradation in vivo. The disclosed hydrogels can prove useful in applications ranging from tissue engineering, drug delivery, and regenerative medicine. Compared to previously disclosed degradable hydrogels, such as those using PEG-based degradable crosslinkers, the presently disclosed hydrogels show advantages in manufacturing due to their increased hydrophobicity and more compact size.

In one aspect, a hydrogel is provided comprising a polymer backbone crosslinked with a first crosslinker containing at least one a moiety of Formula I:

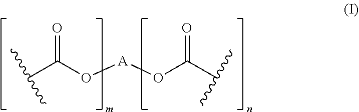

wherein all variables are as defined herein.

In another aspect, a process for synthesizing a hydrogel as described herein comprising reacting a polymer with a first crosslinker comprising at least one moiety of Formula I.

A therapeutic delivery composition is also provided comprising a hydrogel described herein and one or more therapeutic agents. A method of delivering a therapeutic agent to a target site in a subject is also provided, the method comprising administering a therapeutically effective amount of a therapeutic delivery described herein to the target site.

Further provided are cell culture mediums, tissue scaffolds, bioreactors, and wound dressings comprising a hydrogel described herein.

A method of promoting tissue growth in a subject in need thereof is provided, the method comprising:
identifying a target site; and
administering a therapeutically effective amount of a hydrogel described herein to the target site.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIGS. 1A-1K show that hydrolytically degradable microgels can be fabricated by the addition of ester-containing dithiol crosslinkers. FIG. 1A) PEG-4MAL macromer is modified with linear PEG FITC and segmented through a flow-focusing microfluidic chip with a continuous phase containing small dithiol molecules, DTT and EGBMA. This results in monodisperse microgels that can be fluorescently tracked. Scale bar 1 mm. FIGS. 1B-E) Size distribution of microgels based on EGBMA concentration in the oil phase. Inset represents the intensity of individual microgels post-fabrication, demonstrating similar modification of the macromer backbone with the linear PEG-FITC tracker, minimum n=36, pooled from 3 independent microfluidic runs. FIGS. 1F-G) Microgel swelling in an aqueous buffer is directly proportional to the molar concentration of EGBMA linker in the crosslinking phase, minimum n=6 per sample. FIG. 1H) Tracking of released PEG-FITC in solution is dependent on EGBMA concentration in microgels. FIG. 1I) Day 3 images of microgels deformed by an applied pressure in a tapered microcapillary. FIG. 1J) Shear stress vs strain for confined microgels fabricated with varying concentrations of EGBMA after 3 days of incubation in an aqueous buffer, n=6, >60 points total. 1K) Quantification of shear modulus of all microgel formulations after different time exposures to aqueous buffer, n=6. All data presented as average±s.e.m. unless otherwise stated, swelling data analyzed using a mixed-effect model, with Tukey correction for multiple comparisons; Shear modulus was analyzed with a two-way ANOVA with Tukey corrections for multiple comparisons. * p<0.05, ** p<0.01, *** p<0.005, **** p<0.0001.

FIGS. 2A-2D show that microgel co-culture with monocytes does not induce activation in the absence of adhesion cues and inflammatory signals. Cell survival at 48 hr post-incubation does not reveal any changes due to microparticle presence in co-culture. Expression of markers CD45, F4/80, CD206 is equivalent across all groups tested. d-inset represents fold expression of CD206 over all cells expression F4/80 in co-culture. Minimum n=6, all data presented as average±s.e.m. Data was analyzed with one-way ANOVA with Tukey corrections for multiple comparisons.

FIGS. 3A-3E show that degradation of subcutaneous microgel implants is directly proportional to the concentration of the EGBMA linker. FIG. 3A) Scheme of microgel fabrication with a near-infrared PEG linker and injection in a dorsal subcutaneous pocket. Representative images of implant pockets at different time points post-injection and after explant. FIG. 3B) Average normalized radiant efficiency for all formulations throughout the course of a month and after explant (points following vertical dashed line). FIGS. 3C-E) quantification of normalized radiant efficiency at days 0, 9, and 25 post-implantation. All data presented as average±s.d. minimum of n=5 recipients for DTT, n=10 for all other groups. P values calculated using one-way ANOVA with Dunnett multiple comparison analysis, ** p<0.05, *** p<0.0005, **** p<0.0001.

FIGS. 4A-4G show that myeloid cell recruitment and polarization are modulated by the degradation of the synthetic microgel implant at 7 days post-injection. flow cytometry analysis and quantification of myeloid markers CD11b, F4/80, MHCII, and CD206 from subcutaneous implant pockets containing different formulations of nondegradable and degradable microgels. All data presented as average±s.e.m. minimum of n=4 recipients. P values were calculated using one-way ANOVA, correcting for multiple comparisons by controlling the false discovery rate.

FIGS. 5A-5H show that lymphocyte cell recruitment is controlled by the degradation of the synthetic microgel implant 7 days post-injection. Flow cytometry analysis and quantification of lymphocyte markers CD3, CD4, CD8, CD25, and PD-1 from subcutaneous implant pockets containing different formulations of nondegradable and degradable microgels. All data presented as average±s.e.m. minimum of n=4 recipients. P values were calculated using one-way ANOVA, correcting for multiple comparisons by controlling the false discovery rate.

FIGS. 6A-6F show that cytokine responses to implantable synthetic microgels are dynamic and dominated by IFN-γ responses which can altered by the degradation potential of the implantable material. FIG. 6A) Principal component analysis of 32 cytokines measured in implant tissues from animals receiving different synthetic microgel formulations. Arrows color and directions indicate the contribution to each dimension of the PCA. FIG. 6B) Cytokine correlations for all cytokines measured are assessed using Pearson's correlation coefficient. FIG. 6C) Left: hierarchical clustering of cytokines based on Pearson's correlation, dendrogram, and cytokine name denote module membership. FIG. 6D) Correlation plots for all cytokines against IFN-γ. FIGS. 6E-F) Box plots show cytokine concentrations for GM-CSF and IL-4 with raw values plotted on the log 10 scale. Minimum n=6 per recipient. Presented P values are from estimated marginal means (EMM) comparisons.

FIG. 7 shows the hydrolytically susceptible ethylene linkers are used for microparticle crosslinking to fabricate degradable droplet microfluidic based microgels for therapeutic delivery. The tunability and degradability confer by the ester-based degradation in vivo regulates the infiltration of immune cells to the implant site and the host immune polarization.

FIG. 8 is the ¹H NMR spectra of PEG-4MAL macromer and microgels post-fabrication.

FIGS. 9A-9C show the experimental setup for the capillary micromechanics. FIG. 9A) The tapered glass micropipette (Fivephoton Biochemicals) had the following dimensions: tip inner diameter=50 μm; base outer diameter=1.5 mm; length=5.5 cm; taper style=long. A high precision pressure regulator (Elveflow) applied pressure to the micropipette containing the microgel. The micropipette was immersed in 1% BSA to facilitate optimal flow dynamics. The microgel would deform until it reached equilibrium, when the external applied pressure balanced with the internal elastic stress. A microscope (EVOS) under the micropipette tip acquired images (10×), which were subsequently analyzed in ImageJ. FIG. 9B) Microgel geometry in the tapered region. The microgel was in contact with the walls with an average radius, Rband, and average length, Lband. The taper angle is θ. As the pressure, p, increases, Lband increases and Rband decreases. The elastic properties were calculated from these measurements, as previously described (Wyss et al, Soft Matter, 2010). FIG. 9C) Image series of a microgel deforming in response to increasing pressure.

FIG. 10 shows the In vitro cytotoxicity of RAW 264.7 macrophage cells treated with all microgel formulations (degradable, and nondegradables). The graph represents cell viability during a 7-day co-culture determined by Alamar Blue assay. Data represents mean standard deviation of the mean (n=4). No statistical difference found by one-way ANOVA.

FIGS. 11A-11D show the microgel co-culture with monocytes does not induce activation in the absence of adhesion cues and inflammatory signals. 11A-D) Cell survival at 96 hr post-incubation does not reveal any changes due to microparticle presence in co-culture. Expression of markers CD45, F4/80, CD206 is consistent across all groups tested. 11D-inset represents fold expression of CD206 over all cells expression F4/80 in co-culture. All data presented as average±s.e.m, n=3. Data was analyzed with one-way ANOVA with Tukey corrections for multiple comparisons.

FIGS. 12A-12F provide box plots showing cytokine concentrations with raw values plotted on the log 10 scale and estimated marginal means (EMM) comparisons for all time points. n=6 per group.

FIGS. 13A-13D provide box plots showing cytokine concentrations with raw values plotted on the log 10 scale and estimated marginal means (EMM) comparisons for all time points. n=6 per group.

FIG. 14 is the H&E staining at day 30 post-implant of microgels injected in the dorsal subcutaneous space.

FIG. 15 shows the immunohistochemistry assessment of dorsal microgel implants after 30 days post-injection. Samples were stained for pan macrophage marker CD68 (red) and a nuclear marker DAPI (blue). Microgel area represented by white dashed lines. Inset represents a 20× representative image of area surrounding the microgel. Scale bar of inset 20 μm, 10× image 50 μm.

FIGS. 16A and 16B show the degradable hydrogel properties in vitro and in vivo. FIG. 16A) in vivo tracking of hydrogels transplanted into the subcutaneous space of mice. FIG. 16B) IVIS imaging demonstrating localization of microgels, and changes in fluorescence intensity over time.

Like reference symbols in the various drawings indicate like elements.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of 25 steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a cell”, “a tissue”, or “a hydrogel”, includes, but is not limited to, two or more such cells, tissue, or hydrogels, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the 20) sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific composition employed; the duration of the treatment; drugs used in combination or coincidental with the specific composition employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a composition at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the invention (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed composition, by changing the disclosed composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prophylactically effective amount” refers to an amount effective for preventing onset or initiation of a disease or condition.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g. human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as a tissue defect. The effect can be therapeutic in terms of a partial or complete cure of a disease, condition, symptom or adverse effect attributed to the disease, disorder, or condition. The term “treatment” as used herein can include any treatment of a disorder in a subject, particularly a human and can include any one or more of the following: (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

Compounds are described using standard nomenclature. Unless defined otherwise, all technical and scientific terms used herein have the same meaning as is commonly understood by one of skill in the art to which this invention belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the invention and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

“Alkyl” is a straight chain or branched saturated aliphatic hydrocarbon group. In certain embodiments, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one embodiments, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one embodiment, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one embodiment, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one embodiment, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one embodiment, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one embodiment, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one embodiment, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one embodiment, aryl groups are pendant. An example of a pendant ring is a phenyl group substituted with a phenyl group. In one embodiment, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but 25 are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 4, or in some embodiments 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some embodiments from 1 to 3 or from 1 to 2, heteroatoms selected from N, O, S, B, or P, with remaining ring atoms being carbon. In one embodiments, the only heteroatom is nitrogen. In one embodiment, the only heteroatom is oxygen. In one embodiment, the only heteroatom is sulfur. Monocyclic heteroaryl groups typically have from 5 to 6 ring atoms. In some embodiments, bicyclic heteroaryl groups are 8- to 10-membered heteroaryl groups, that is groups containing 8 or 10 ring atoms in which one 5-, 6-, or 7-membered aromatic ring is fused to a second aromatic or non-aromatic ring, wherein the point of attachment is the aromatic ring. When the total number of S and O atoms in the heteroaryl group excess 1, these heteroatoms are not adjacent to one another. In one embodiment, the total number of S and O atoms in the heteroaryl group is not more than 2. In another embodiment, the total number of S and O atoms in the heteroaryl group is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

In one aspect, a hydrogel is provided comprising a polymer backbone crosslinked with a first crosslinker containing at least one a moiety of Formula I:

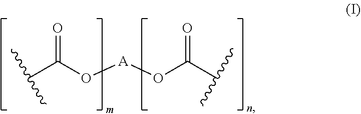

wherein:
m and n are independently 1 or 2;
A is C₂-C₁₀ alkyl; and

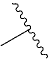

is a point of attachment for the moiety within the first crosslinker.

In some embodiments of Formula I, m is 1. In some embodiments of Formula I, m is 2. In some embodiments of Formula I, n is 1. In some embodiments of Formula I, n is 2.

In some embodiments of Formula I, A is selected from C₂ alkyl, C₃ alkyl, C₄ alkyl, C₅ alkyl, C₆ alkyl, C₇ alkyl, C₈ alkyl, C₉ alkyl, or C₁₀ alkyl. In some embodiments of Formula I, A is selected from ethyl, n-propyl, isopropyl, n-butyl, isobutyl, sec-butyl, t-butyl, n-pentyl, isopentyl, tert-pentyl, neopentyl, n-hexyl, 2-methylpentyl, 3-methylpentyl, 2,2-dimethylbutyl, and 2,3-dimethylbutyl.

In some embodiments, the moiety of Formula I may be selected from:

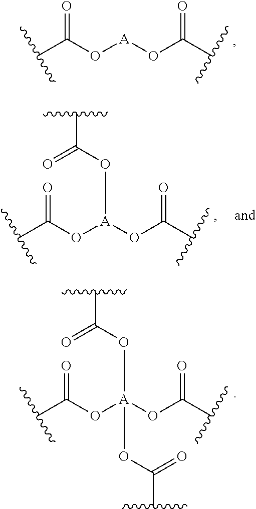

The polymer backbone may be comprised of any polymer or combination of polymers which finds use in the preparation of a hydrogel. As is known in the art, a hydrogel is a polymer network formed by crosslinking one or more multifunctional molecules or polymers. The resulting polymeric network is hydrophilic and swells in an aqueous environment thus forming a gel-like material, i.e., hydrogel. Typically, a hydrogel comprises a backbone bonded to a crosslinking agent (such as the first crosslinker described herein).

Hydrogels are characterized by their water insolubility, hydrophilicity, high water absorbability and swellable properties. The molecule components, units or segments of a hydrogel are characterized by a significant portion of hydrophilic components, units, or segments, such as segments capable of hydrogen bonding or having ionic species or dissociable species, such as acids (e.g., carboxylic acids, phosphonic acids, sulfonic acids, sulfinic acids, phosphinic acids, etc.), bases (e.g., amine groups, proton accepting groups, etc.), or other groups that develop ionic properties when immersed in water (e.g., sulfonamides). Acryloyl groups (and to a lesser degree methacryloyl groups) and the class of acrylic polymers or polymer chaings containing or terminated with oxyalkylene units (such as polyoxyethylene chains and polyoxyethylene/polyoxypropylene copolymer chains) are also well recognized as hydrophilic segments that may be present within hydrophilic polymers. Representative water insoluble polymeric compositions are provided below, although the entire class of hydrogel materials known in the art may be used to varying degrees. The polymers set forth below and containing acidic groups can be, as an option, partially or completely neutralized with alkali metal bases, either in monomer or the polymer or both.

Some representative polymers which may comprise the polymer backbone include, but are not limited to: polyacrylic acid, polymethacrylic acid, polymaleic acid, copolymers thereof, and alkali metal and ammonium salts thereof; graft copolymers of starch and acrylic acid, starch and saponified acrylonitrile, starch and saponified ethyl acrylate, and acrylate-vinyl acetate copolymers saponified; polyvinyl alcohol, polyvinylpyrrolidone, polyvinyl alkylether, polyethylene oxide, polyacrylamide, and copolymers thereof; copolymers of maleic anhydride and alkyl vinylethers; and saponified starch graft copolymers of acrylonitrile, acrylate esters, vinyl acetate, and starch graft copolymers of acrylic acid, methacrylic acid, and maleic acid.

In some embodiments, the polymer backbone may comprise a biopolymer. In some embodiments, the biopolymer may have been functionalized or modified in such a manner that provides a functionality enabling crosslinking with the first crosslinker. Representative examples of biopolymers which may be used include, but are not limited to, collagen, gelatin, fibrin, hyaluronic acid, elastin, pectin, agarose, glycoaminoglycans, alginates, cellulose, DNA, RNA, or functionalized derivatives thereof.

In some embodiments, the polymer backbone comprises a poly(ethylene glycol) or a functionalized derivative thereof. Representative examples of such polymer backbones may be formed from polymers including, but not limited to, poly(ethylene glycol) (PEG), poly(ethylene glycol)-di-acrylate (PEG-DA), multi-arm poly(ethylene glycol)-acrylate (PEG-Ac), poly(ethylene glycol)-dithiol (PEG-diSH), poly(ethylene glycol)divinyl sulfone (PEG-diVS), multi-arm poly(ethylene glycol)vinyl sulfone (PEG-VS), poly(ethylene glycol)-di-methacrylate (PEG-DMA), multi-arm poly(ethylene glycol)-methacrylate (PEG-Mac), poly(ethylene glycol)-di-allyl ether (PEG-diAE), multi-arm poly(ethylene glycol)-allyl ether (PE-AD), poly(ethylene glycol)-di-vinyl ether (PEG-diVE), multi-arm poly(ethylene glycol)-vinyl ether (PEG-VE), poly(ethylene glycol)-di-maleimide (PEG-diMI), multi-arm poly(ethylene glycol)-maleimide (PEG-MI), poly(ethylene glycol)-di-norborene, multi-arm poly(ethylene glycol)norborene, poly(ethylene glycol-vinyl carbonate, multi-arm poly(ethylene glycol)-vinyl carbonate, and polyethylene glycol oligofumarate.

In some particular embodiments, the polymer backbone may be formed from a multi-arm poly(ethylene glycol)-maleimide.

The above exemplary polymers may be cross-linked either during polymerization or after polymerization using a first crosslinker as described herein and optionally one or more additional crosslinkers. The crosslinking may be performed using methods known to those skilled in the art, such as for example via initiation in the presence of radiation of via a radical initiator.

The first crosslinker comprises at least two moieties capable of reacting with the polymer backbone. The polymer backbone itself has active groups available to react with the at least two moieties of the first crosslinking to form covalent linkages. It is generally understood that the presence of the moiety of Formula I within the first crosslinker, once crosslinked, leads to the observed properties of hydrolytic degradation for the hydrogels provided herein.

In some embodiments, the first crosslinker comprises a compound of Formula II:

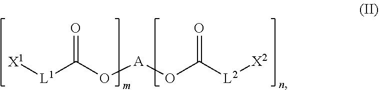

wherein:
X¹ and X² are independently selected at each occurrence from a moiety capable of reacting with the polymer backbone;
L¹ and L² are independently selected at each occurrence from a linking moiety; and
m, n, and A are defined as in claim 1.

In some embodiments of Formula II, m is 1. In some embodiments of Formula II, m is 2. In some embodiments of Formula II, n is 1. In some embodiments of Formula II, n is 2.

In some embodiments of Formula II, A is selected from C₂ alkyl, C₃ alkyl, C₄ alkyl, C₅ alkyl, C₆ alkyl, C₇ alkyl, C₈ alkyl, C₉ alkyl, or C₁₀ alkyl. In some embodiments of Formula II, A is selected from ethyl, n-propyl, isopropyl, n-butyl, isobutyl, sec-butyl, t-butyl, n-pentyl, isopentyl, tert-pentyl, neopentyl, n-hexyl, 2-methylpentyl, 3-methylpentyl, 2,2-dimethylbutyl, and 2,3-dimethylbutyl.

In some embodiments, the compound of Formula II is selected from:

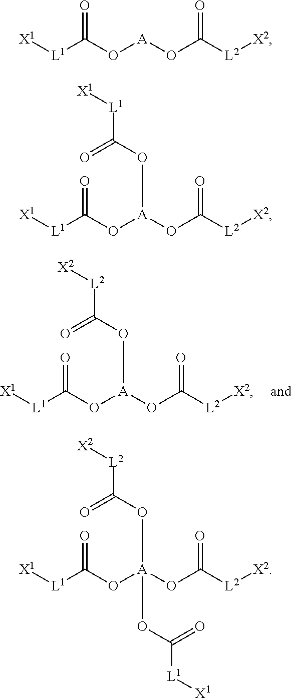

X¹ and X² may each independently be any suitable moiety of functionality which is capable of reacting with an active group or moiety as found in the polymer backbone. Representative examples of such groups include, but are not limited to, halo, hydroxy, amino, thiol, carboxylic acid, ester, or the like. In some embodiments, the group X¹ and X² may each independently comprise a group which is polymerizable, such as an oxiranyl, acryloyl, or methacryloyl group, or the like. In particular embodiments, X¹ and X² are each —SH.

L¹ and L² may each independently comprise a bond or any other suitable linking moiety which covalently links the moieties X¹ and X² to the corresponding carbonyl group to which it is attached. In some embodiments, L¹ and L² may be independently selected from C₁-C₆ alkyl, C₁-C₆ heteroalkyl, C₁-C₆ haloalkyl, C₂-C₆ alkenyl, C₂-C₆ alkynyl, C₃-C₆ cycloalkyl, 3- to 8-membered monocyclic or bicyclic heterocycle, 6- to 10-membered monocyclic or bicyclic aryl, 5- to 10-membered monocyclic or bicyclic heteroaryl, or any suitable combination thereof, each of which may be optionally substituted as described herein.

In some embodiments, L¹ and L² are each independently selected from C₁-C₁₀ alkyl, for example, methylene, ethylene, propylene, or butylene. In particular embodiments, L¹ and L² are each methylene.

In particular embodiments, the first crosslinker comprises ethylene glycol bis(mercaptoacetate).

In some embodiments, the polymer backbone is further crosslinked with a second crosslinker. In such embodiments, the second crosslinker is typically hydrolytically stable, i.e., does not contain the moiety of Formula I as found within the first crosslinker or any other moiety which may be hydrolytically cleaved under conditions for which the hydrogel is intended to be used. In particular embodiments, the second crosslinker may comprise dithiothreitol (DTT).

In embodiments containing a second crosslinker, degradation of the hydrogel may be tunable by varying the molar ratio of the first crosslinker to the second crosslinker. In some embodiments, the molar ratio of the first crosslinker to the second crosslinker can range from about 100:1 to about 1:100, for example about 90:1, about 80:1, about 70:1, about 60:1, about 50:1, about 40:1, about 30:1, about 25:1, about 20:1, about 15:1, about 10:1, about 5:1, about 4:1, about 3:1, about 2:1, about 1:1, about 1:2, about 1:3, about 1:4, about 1:5, about 1:10, about 1:15, about 1:20, about 1:25, about 1:30, about 1:40, about 1:50, about 1:60, about 1:70, about 1:80, about 1:90, or about 1:100. It may be readily understood to the skilled reader that a higher molar ratio of the first crosslinker to the second crosslinker would lead to increased hydrolytic degradation and shortened degradation times, while a lower molar ratio of the first crosslinker to the second crosslinker would lead to lower hydrolytic degradation and longer degradation times.

The degradation products of the hydrogel should be substantially biocompatible, i.e., will not substantially adversely affect the body, tissue, or cells of the living subject or otherwise, either at the site where the hydrogel is placed or in any other parts of the living subject. Methods for assessing the biocompatibility of a material are well known.

In some embodiments, the hydrogels described herein may contain a bioactive agent capable of modulating a function and/or characteristic of a cell. For example, the bioactive agent may be capable of modulating a function and/or characteristic of a cell that is dispersed on or within the hydrogel. Alternatively or additionally, the bioactive agent may be capable of modulating a function and/or characteristic of an endogenous cell surrounding a hydrogel implanted in a tissue defect, for example, and guide the cell into the defect. The at least one bioactive agent can include polynucleotides and/or polypeptides encoding or comprising, for example, transcription factors, differentiation factors, growth factors, or combinations thereof. The at least one bioactive agent can also include any agent capable of promoting tissue formation, destruction, and/or targeting a specific disease state (for example, cancer). Representative examples of such bioactive agents include, but are not limited to, chemotactic agents, various proteins (such as short term peptides, bone morphogenic proteins, collagen, glycoproteins, and lipoprotein), cell attachment mediators, biologically active ligands, integrin binding sequence, various growth and/or differentiation agents and fragments thereof (such as epidermal growth factor (EGF), hepatocyte growth factor (HGF), vascular endothelial growth factors (VEGF), fibroblast growth factors (e.g., bFGF), platelet derived growth factors (PDGF), insulin-like growth factor (e.g., IGF-1, IGF-II) and transforming growth factors (e.g., TGF-β I-III)), parathyroid hormone, parathyroid hormone related peptide, bone morphogenic proteins (e.g., BMP-2, BMP-4, BMP-6, BMP-7, BMP-12, BMP-13, BMP-14), transcription factors, such as sonic hedgehog, growth differentiation factors (e.g., GDF5, GDF6, GDF8), recombinant human growth factors (such as MP52 and MP-52 variant rhGDF-5), cartilage-derived morphogenic proteins (CDMP-1, CDMP-2, CDMP-3), small molecules that affect the upregulation of specific growth factors, tenascin-C, hyaluronic acid, chondroitin sulfate, fibronectin, decorin, thromboelastin, thrombin-derived peptides, heparin-binding domains, heparin, heparan sulfate, polynucleotides, DNA fragments, DNA plasmids, MMPs, TIMPs, interfering RNA molecules, such as siRNAs, oligonucleotides, proteoglycans, glycoproteins, glycosaminoglycans, and DNA encoding for shRNA.

In some embodiments, the hydrogels described herein may contain a therapeutic agent which may be used in treating of a condition or disorder in a subject in need of such treatment. The term “therapeutic agent” includes any synthetic or naturally occurring biologically active compound or composition of matter which, when administered to an organism (either human or a nonhuman animal), induces a desired pharmacologic, immunogenic, and/or physiologic effect by local and/or systemic action. The term therefore encompasses those compounds or chemicals traditionally regard as drugs, vaccines, and biopharmaceuticals including molecules such as proteins, peptides, hormones, nucleic acids, gene constructs and the like. Examples of therapeutic agents are described in well-known literature references such as the Merk Index (14^{th} Edition), the Physician's Desk Reference (64^{th} Edition), and The Pharmacological Basis of Therapeutics (12^{th} Edition), and they include, without limitation, medicaments; vitamins; mineral supplements, substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiandrenergics, antiarrhythmics, antihypertensive agents, hormones, and nutrients, antiarthritics, antiasthmatic agents, anticonvulsants, antihistamines, antinauseants, antineoplastics, antipruritics, antipyretics, antispasmodics, cardiovascular preparations (including calcium channel blockers, beta blockers, and beta-agonists), antihypertensives, diuretics, vasodilators, central nervous system stimulants, cough and cold preparations, decongestants, diagnostics, bone growth stimulants and bone resorption inhibitors, immunosuppressives, muscle relaxants, psychostimulants, sedatives, tranquilizers, proteins, peptides, and fragments thereof (whether naturally occurring, chemically synthesized or recombinantly produced), and nucleic acid molecules (polymeric forms of two or more nucleotides, either ribonucleotides (RNA) or deoxyribonucleotides (DNA) including both double and single-stranded molecules, gene constructs, expression vectors, antisense molecules and the like), small molecules and other biologically active macromolecules such as, for examples, proteins and enzymes. The agent may be a biologically active agent used in medical, including veterinary, applications and in agriculture, such as with plants, as well as other areas.

The hydrogel can be injectable and/or implantable, or can be in the form of a membrane, sponge, gel, solid scaffold, spun fiber, woven or unwoven mesh, nanoparticle, microparticle, or any other desirable configuration.

In another aspect, the hydrogel can include at least one cell dispersed on or within the hydrogel. For example, cells can be entirely or partly encapsulated within the hydrogel. Cells can include, for example, any progenitor cell, such as a totipotent stem cell, a pluripotent stem cell, or a multipotent stem cell, as well as any of their lineage descendent cells, including more differentiated cells. The cells can be autologous, xenogeneic, allogeneic, and/or syngeneic. Where the cells are not autologous, it may be desirable to administer immunosuppressive agents in order to minimize immunorejection. The cells employed may be primary cells, expanded cells, or cell lines, and may be dividing or non-dividing cells. Cells may be expanded ex vivo prior to introduction into or onto the hydrogel. For example, autologous cells can be expanded in this manner if a sufficient number of viable cells cannot be harvested from the host subject. Alternatively or additionally, the cells may be pieces of tissue, including tissue that has some internal structure. The cells may be primary tissue explants and preparations thereof, cell lines (including transformed cells), or host cells.

In some embodiments, a cell can refer to any progenitor cell, such as totipotent stem cells, pluripotent stem cells, and multipotent stem cells, as well as any of their lineage descendent cells, including more differentiated cells. The terms “stem cell” and “progenitor cell” are used interchangeable herein. The cells can be derived from embryonic, fetal, or adult tissues. Examples of progenitor cells can include totipotent stem cells, multipotent stem cells, mesenchymal stem cells (MSCs), hematopoietic stem cells, neuronal stem cells, pancreatic stem cells, cardiac stem cells, embryonic stem cells, embryonic germ cells, neural crest stem cells, kidney stem cells, hepatic stem cells, lung stem cells, hemangioblast cells, and endothelial progenitor cells. Additional exemplary progenitor cells can include de-differentiated chondrogenic cells, chondrogenic cells, cord blood stem cells, multi-potent adult progenitor cells, myogenic cells, osteogenic cells, tendogenic cells, ligamentogenic cells, adipogenic cells, and dermatogenic cells.

The hydrogel can be formed with at least one cell and/or bioactive agent. For example, a plurality of cells may be dispersed in a substantially uniform manner on or within the hydrogel, or, alternatively, dispersed such that different densities and/or spatial distributions of different or the same cells are dispersed within different portions of the hydrogel. The cells may be seeded before or after crosslinking of the polymer backbone. Alternatively, the hydrogel can be incubated in a solution of at least one bioactive agent after crosslinking of the polymer backbone.

Generally, cells be introduced into the hydrogel in vitro or in vivo. Cells may be mixed with the hydrogel and cultured in an adequate growth (or storage) medium to ensure cell viability. If the hydrogel is to be implanted for use in vivo after in vitro seeding, for example, sufficient growth medium may be supplied to ensure cell viability during in vitro culture prior to in vivo application. Once the hydrogel has been implanted, the nutritional requirements of the cells can be met by the circulating fluids of the host subject.

Any available method may be employed to introduce the cells into the hydrogel. For example, cells may be injected into the hydrogel (such as in combination with growth medium) or may be introduced by other means, such as pressure, vacuum, osmosis, or manual mixing. Alternatively or additionally, cells may be layers on the hydrogel, or the hydrogel may be dipped into a cell suspension and allowed to remain their under conditions and for a time sufficient for the cells to incorporate within or attach to the hydrogel. Generally, it is desirable to avoid excessive manual manipulation of the cells in order to minimize cell death during the impregnation procedure. For example, in some situations it may not be desirable to manually mix or knead the cells with the hydrogel; however, such an approach may be useful in those cases in which a sufficient number of cells will survive the procedure. Cells can also be introduced into the hydrogel in vivo simply by placing the hydrogel in the subject adjacent to a source of desired cells. Bioactive agents may be released from the hydrogel, if contained therein, which may also recruit local cells, cells in the circulation, or cells at a distance from the implantation or injection site.

The number of cells introduced into the hydrogel will vary based on the intended application of the hydrogel and the type of cell used. For example, when dividing autologous cells are being introduced by injection or mixing into the hydrogel, a lower number of cells can be used. Alternatively, where non-dividing cells are being introduced by injection or mixing into the hydrogel, a larger number of cells may be required. The hydrogel may either be in a hydrated or lyophilized state prior to the addition of cells. For example, the hydrogel can be in a lyophilized state before the addition of cells is done to rehydrate and populate the hydrogel with cells.

The hydrogels described herein can be used in a variety of biomedical applications, including tissue engineering, drug delivery applications, and regenerative medicine. In one example, a hydrogel described herein can be used to promote tissue growth in a subject. One step of the method can include identifying a target site. The target site can comprise a tissue defect in which promotion of new tissue is desired. The target site can also comprise a disease location (for example, a tumor). Methods for identifying tissue defects and disease locations are known in the art and can include, for example, various imaging modalities, such as CT, MRI, and X-ray. After identifying a target site, the hydrogel can be administered to the target site. Next, the hydrogel may be loaded into a syringe or other similar device and injected or implanted into the tissue defect. Upon injection or implantation into the tissue defect, the hydrogel can be formed into the shape of the tissue defect using tactile means. Alternatively, the hydrogel may be formed into a specific shape prior to implantation into the subject. After implanting, the cells can begin to migrate from the hydrogel into the tissue defect, express growth and/or differentiation factors, and/or promote cell expansion and differentiation. Additionally, the presence of the hydrogel in the tissue defect may promote migration of endogenous cells surrounding the tissue defect into the hydrogel. Once implanted, the moiety of Formula I can be hydrolyzed. Hydrolysis of this moiety can occur at a controlled rate and lead to controlled degradation of the hydrogel. This degradation can create space for cell growth and deposition of a new extracellular matrix to replace the hydrogel.

As used herein, the term “tissue” can refer to an aggregate of cells having substantially the same function and/or form in a multicellular organism. “Tissue” is typically an aggregate of cells of the same origin, but may be an aggregate of cells of different origins. The cells can have substantially the same or substantially different function, and may be of the same or different type. “Tissue” can include, but is not limited to, an organ, a part of an organ, bone, cartilage, skin, neuron, axon, blood vessel, cornea, muscle, fascia, brain, prostate, breast, endometrium, lung, pancreas, small intestine, blood, liver, testes, ovaries, cervix, colon, stomach, esophagus, spleen, lymph node, bone marrow, kidney, peripheral blood, embryonic, or ascite tissue.

Kits for practicing the methods described herein are further provided. By “kit” is intended any manufacture (e.g., a package or a container) comprising at least one reagent, e.g., any one of the compositions described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

Also disclosed are kits that comprise a composition disclosed herein in one or more containers. The disclosed kits can optionally include pharmaceutically acceptable carriers and/or diluents. In one embodiment, a kit includes one or more other components, adjuncts, or adjuvants as described herein. In one embodiment, a kit includes instructions or packaging materials that describe how to administer a composition of the kit. Containers of the kit can be of any suitable material, e.g., glass, plastic, metal, etc., and of any suitable size, shape, or configuration. In one embodiment, a composition agent disclosed herein is provided in the kit as a solid. In another embodiment, a composition disclosed herein is provided in the kit as a liquid or solution. In one embodiment, the kit comprises an ampoule or syringe containing a composition described herein in liquid or solution form.

The present disclosure also provides the following embodiments of the invention provided herein:
Embodiment 1. A hydrogel comprising a polymer backbone crosslinked with a first crosslinker containing at least one a moiety of Formula I:

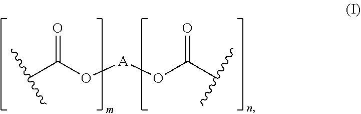

wherein:
m and n are independently 1 or 2;
   A is C₂-C₁₀ alkyl; and

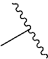

is a point of attachment for the moiety within the first crosslinker.
Embodiment 2. The hydrogel of embodiment 1, wherein m is 1.
Embodiment 3. The hydrogel of embodiment 1, wherein m is 2.
Embodiment 4. The hydrogel of any one of embodiments 1-3, wherein n is 1.
Embodiment 5. The hydrogel of any one of embodiments 1-3, wherein n is 2.
Embodiment 6. The hydrogel of any one of embodiments 1-5, wherein A is selected from C₂-C₈ alkyl, C₂-C₆ alkyl, and C₂-C₄ alkyl.
Embodiment 7. The hydrogel of any one of embodiments 1-6, wherein A is C₂ alkyl.
Embodiment 8. The hydrogel of any one of embodiments 1-7, wherein the polymer backbone comprises a poly(ethylene glycol) or a functionalized derivative thereof.
Embodiment 9. The hydrogel of any one of embodiments 1-8, wherein the polymer backbone comprises a polymer selected from poly(ethylene glycol) (PEG), poly(ethylene glycol)-di-acrylate (PEG-DA), multi-arm poly(ethylene glycol)-acrylate (PEG-Ac), poly(ethylene glycol)-dithiol (PEG-diSH), poly(ethylene glycol)divinyl sulfone (PEG-diVS), multi-arm poly(ethylene glycol)vinyl sulfone (PEG-VS), poly(ethylene glycol)-di-methacrylate (PEG-DMA), multi-arm poly(ethylene glycol)-methacrylate (PEG-Mac), poly(ethylene glycol)-di-allyl ether (PEG-diAE), multi-arm poly(ethylene glycol)-allyl ether (PE-AD), poly(ethylene glycol)-di-vinyl ether (PEG-diVE), multi-arm poly(ethylene glycol)-vinyl ether (PEG-VE), poly(ethylene glycol)-di-maleimide (PEG-diMI), multi-arm poly(ethylene glycol)-maleimide (PEG-MI), poly(ethylene glycol)-di-norborene, multi-arm poly(ethylene glycol)norborene, poly(ethylene glycol-vinyl carbonate, multi-arm poly(ethylene glycol)-vinyl carbonate, and polyethylene glycol oligofumarate, or combinations thereof.
Embodiment 10. The hydrogel of any one of embodiments 1-9, wherein the polymer backbone comprises multi-arm poly(ethylene glycol)-maleimide.
Embodiment 11. The hydrogel of any one of embodiments 1-10, wherein the first crosslinker comprises m+n moieties capable of reacting with the polymer backbone, wherein m and n are as defined in embodiment 1.
Embodiment 12. The hydrogel of any one of embodiments 1-11, wherein the first crosslinker comprises a compound of Formula II:

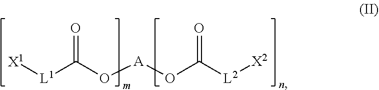

wherein:
X¹ and X² are independently selected at each occurrence from a moiety capable of reacting with the polymer backbone;
L¹ and L² are independently selected at each occurrence from a linking moiety; and
m, n, and A are defined as in embodiment 1.
Embodiment 13. The hydrogel of embodiment 12, wherein X¹ and X² are each —SH.
Embodiment 14. The hydrogel of embodiment 12 or embodiment 13, wherein L¹ and L² are independently selected at each occurrence from C₁-C₁₀ alkyl.
Embodiment 15. The hydrogel of any one of embodiments 1-14, wherein the first crosslinker comprises ethylene glycol bis(mercaptoacetate).
Embodiment 16. The hydrogel of any one of embodiments 1-15, wherein the first crosslinker is hydrolytically degradable.
Embodiment 17. The hydrogel of any one of embodiments 1-16, wherein the polymer backbone is further crosslinked with a second crosslinker.
Embodiment 18. The hydrogel of embodiment 17, wherein the second crosslinker is hydrolytically stable.
Embodiment 19. The hydrogel of embodiment 17 or embodiment 18, wherein the second crosslinker comprises dithiothreitol (DTT).
Embodiment 20. The hydrogel of any one of embodiments 17-19, wherein degradation of the hydrogel is tunable by varying the molar ratio of the first crosslinker to the second crosslinker.
Embodiment 21. The hydrogel of any one of embodiments 1-20, wherein the hydrogel is injectable and/or implantable.
Embodiment 22. The hydrogel of any one of embodiments 1-21, wherein the hydrogel is in the form of a membrane, sponge, gel, solid scaffold, spun fiber, woven or unwoven mesh, nanoparticle, or microparticle.
Embodiment 23. The hydrogel of any one of embodiments 1-22, further comprising at least one cell.
Embodiment 24. A process for synthesizing a hydrogel of any one of embodiments 1-23 comprising reacting a polymer with a first crosslinker comprising at least one moiety of

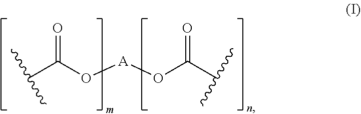

wherein all variables are as defined in embodiment 1.
Embodiment 25. The process of embodiment 24, wherein the first crosslinker comprises a compound of Formula II:

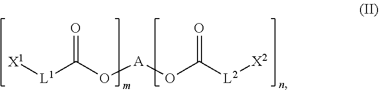

wherein:
X¹ and X² are independently selected at each occurrence from a moiety capable of reacting with the polymer backbone;
L¹ and L² are independently selected at each occurrence from a linking moiety; and
m, n, and A are defined as in embodiment 1.
Embodiment 26. The process of embodiment 24 or embodiment 25, wherein the first crosslinker comprises ethylene glycol bis(mercaptoacetate).
Embodiment 27. The process of any one of embodiments 24-26, further comprising reacting the hydrogel with a second crosslinker, wherein the second crosslinker is hydrolytically stable.
Embodiment 28. The process of embodiment 27, wherein the second crosslinker comprises dithiothreitol (DTT).
Embodiment 29. A therapeutic delivery composition comprising a hydrogel of any one of embodiments 1-23 and one or more therapeutic agents.
Embodiment 30. The therapeutic delivery composition of embodiment 29, wherein the one or more therapeutic agents may be selected from a cell, a protein, an antibody, a nucleic acid, a growth factor, or a drug.
Embodiment 31. A cell culture medium comprising a hydrogel of any one of embodiments 1-23.
Embodiment 32. A tissue scaffold comprising a hydrogel of any one of embodiments 1-23.
Embodiment 33. A bioreactor comprising a hydrogel of any one of embodiments 1-23.
Embodiment 34. A wound dressing comprising a hydrogel of any one of embodiments 1-23.
Embodiment 35. A method of promoting tissue growth in a subject in need thereof, comprising:
   identifying a target site; and
   administering a therapeutically effective amount of a hydrogel of any one of embodiments 1-23 to the target site.
Embodiment 36. The method of embodiment 35, wherein the target site comprises a tissue defect in which promotion of new tissue is desired.
Embodiment 37. The method of embodiment 35 or embodiment 36, wherein the target site is identified using an imaging modality.
Embodiment 38. The method of embodiment 37, wherein the imaging modality is selected from CT, MRI, or X-Ray.
Embodiment 39. The method of any one of embodiments 35-38, wherein the hydrogel is injected or implanted into the target site.
Embodiment 40. A method of delivering a therapeutic agent to a target site in a subject, the method comprising administering a therapeutically effective amount of a therapeutic delivery composition of embodiment 29 or embodiment 30 to the target site.
Embodiment 41. The method of embodiment 40, wherein the target site is associated with a disease state or condition.
Embodiment 42. The method of embodiment 40 or embodiment 41, wherein the target site is a tumor.
Embodiment 43 The method of any one of embodiments 40-42, wherein the target site is identified using an imaging modality.
Embodiment 44. The method of embodiment 43, wherein the imaging modality is selected from CT, MRI, or X-Ray.
Embodiment 45. The method of any one of embodiments 40-44, wherein the hydrogel is injected or implanted into the target site.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are put forth so as to provide those of ordinary skill in the art with a complete disclosure and description of how the compositions, articles, and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Example 1. Hydrolytically Degradable Microgels with Tunable Mechanical Properties Modulate the Host Immune Response

The application of ester-containing linkers offers a degradation mechanism based on hydrolytic cleavage of the ester bond. Degradation can be controlled by polymer content, macromer molecular weight, crosslinking density, and hydrophobicity of the ester labile linker (Jo, Y. S.; Gantz, J.; Hubbell, J. A.; Lutolf, M. P. Tailoring Hydrogel Degradation and Drug Release via Neighboring Amino Acid Controlled Ester Hydrolysis. Soft Matter 2009, 5 (2), 440-446 and Zustiak, S. P.; Leach, J. B. Hydrolytically Degradable Poly(Ethylene Glycol) Hydrogel Scaffolds with Tunable Degradation and Mechanical Properties. Biomacromolecules 2010, 11 (5), 1348-1357). In contrast to hydrogels with enzymatic degradation, the hydrogels developed through this approach are degradable through hydrolysis, allowing for consistent degradation profiles dependent solely on the adjustable physical, mechanical, and chemical properties of the hydrogel (Jo, Y. S.; Gantz, J.; Hubbell, J. A.; Lutolf, M. P. Tailoring Hydrogel Degradation and Drug Release via Neighboring Amino Acid Controlled Ester Hydrolysis. Soft Matter 2009, 5 (2), 440-446). Whereas bulk gels have previously been engineered with hydrolytically degradable crosslinkers (Jo, Y. S.; Gantz, J.; Hubbell, J. A.; Lutolf, M. P. Tailoring Hydrogel Degradation and Drug Release via Neighboring Amino Acid Controlled Ester Hydrolysis. Soft Matter 2009, 5 (2), 440-446 and Hunckler, M. D.; Medina, J. D.; Coronel, M. M.; Weaver, J. D.; Stabler, C. L.; García, A. J. Linkage Groups within Thiol-Ene Photoclickable PEG Hydrogels Control In Vivo Stability. Advanced Healthcare Materials 2019, 8 (14), 1900371), this modality has yet to be translated to microgels fabricated through microfluidic-based polymerization.

The ability to incorporate degradability into the hydrogel network constitutes a major advantage for regenerative medicine and immunoengineering applications, as material persistence and mechanical properties will regulate the tissue response to the implant. The immune response to a biomaterial will ultimately determine the fate of the implanted material, whether it is integrated into the local tissue or walled off by the foreign body response (FBR). Initially, following biomaterial implantation, an inflammatory type 1 injury response will develop near the material, driven by pro-inflammatory mediators IFNγ and TNFα. Pro-regenerative biomaterials then drive a transition to a type 2 immune response, promoting M2 (CD206⁺) macrophage polarization and T helper 2 cells infiltration via IL-4 signaling (Developing a pro-regenerative biomaterial scaffold microenvironment requires T helper 2 cells https://www.science.org/doi/10.1126/science.aad9272 (accessed 2022-02-11)). On the other hand, the host response to synthetic implants is typically characterized by a foreign body reaction that primarily activates mononuclear phagocytes (Sussman, E. M.; Halpin, M. C.; Muster, J.; Moon, R. T.; Ratner, B. D. Porous Implants Modulate Healing and Induce Shifts in Local Macrophage Polarization in the Foreign Body Reaction. Ann Biomed Eng 2014, 42 (7), 1508-1516 and Mikos, A. G., et al. Host response to tissue engineered devices Adv Drug Deliv Rev 1998, 33(1-2): 111-139) and other cells involved in executing type 3 immune responses (Chung, L.; Maestas, D. R.; Lebid, A.; Mageau, A.; Rosson, G. D.; Wu, X.; Wolf, M. T.; Tam, A. J.; Vanderzee, I.; Wang, X.; Andorko, J. I.; Zhang, H.; Narain, R.; Sadtler, K.; Fan, H.; Čiháková, D.; Le Saux, C. J.; Housseau, F.; Pardoll, D. M.; Elisseeff, J. H. Interleukin 17 and Senescent Cells Regulate the Foreign Body Response to Synthetic Material Implants in Mice and Humans. Sci Transl Med 2020, 12 (539), eaax3799). Activated macrophages and Th17 cells secrete TGFβ and other factors that recruit fibroblasts, promote their differentiation to myofibroblasts, and drive fibrosis at the implant surface. Recently, more complex interactions between different immune populations of varying phenotypes have been implicated in response to biomaterials (Doloff, J. C.; Veiseh, O.; de Mezerville, R.; Sforza, M.; Perry, T. A.; Haupt, J.; Jamiel, M.; Chambers, C.; Nash, A.; Aghlara-Fotovat, S.; Stelzel, J. L.; Bauer, S. J.; Neshat, S. Y.; Hancock, J.; Romero, N. A.; Hidalgo, Y. E.; Leiva, I. M.; Munhoz, A. M.; Bayat, A.; Kinney, B. M.; Hodges, H. C.; Miranda, R. N.; Clemens, M. W.; Langer, R. The Surface Topography of Silicone Breast Implants Mediates the Foreign Body Response in Mice, Rabbits and Humans. Nat Biomed Eng 2021, 5 (10), 1115-1130 and Witherel, C. E.; Sao, K.; Brisson, B. K.; Han, B.; Volk, S. W.; Petrie, R. J.; Han, L.; Spiller, K. L. Regulation of Extracellular Matrix Assembly and Structure by Hybrid MI/M2 Macrophages. Biomaterials 2021, 269, 120667). However, this characterization pertains mostly to bulk hydrogel implants with relatively little research on the effect of microgel implantation and degradation on local tissue responses.

Herein, a fabrication approach based on flow-focusing droplet generation is presented that produces monodisperse hydrolytically degradable microgels with modular mechanical and degradation profiles dependent on the introduction of a labile ethylene linker, ethylene glycol bis(mercaptoacetate)(EGBMA). It is demonstrated that controlled hydrogel degradation profiles can be achieved by tuning the ester concentration in the hydrogel microparticle via the addition of varying molar concentrations of EGBMA to a nondegradable linker in the continuous flow phase. The addition of EGBMA did not influence macrophage polarization in vitro while it promoted degradation in vivo. Additionally, the effects of degradability on tissue responses is characterized to the microgel suspension implant. It is demonstrated that control over the degradation profile of the microgel suspension can modulate type 1 immune responses to the implant.

### Addition of Ester-Containing Dithiol Molecules Generates Hydrolytically Degradable Microgels

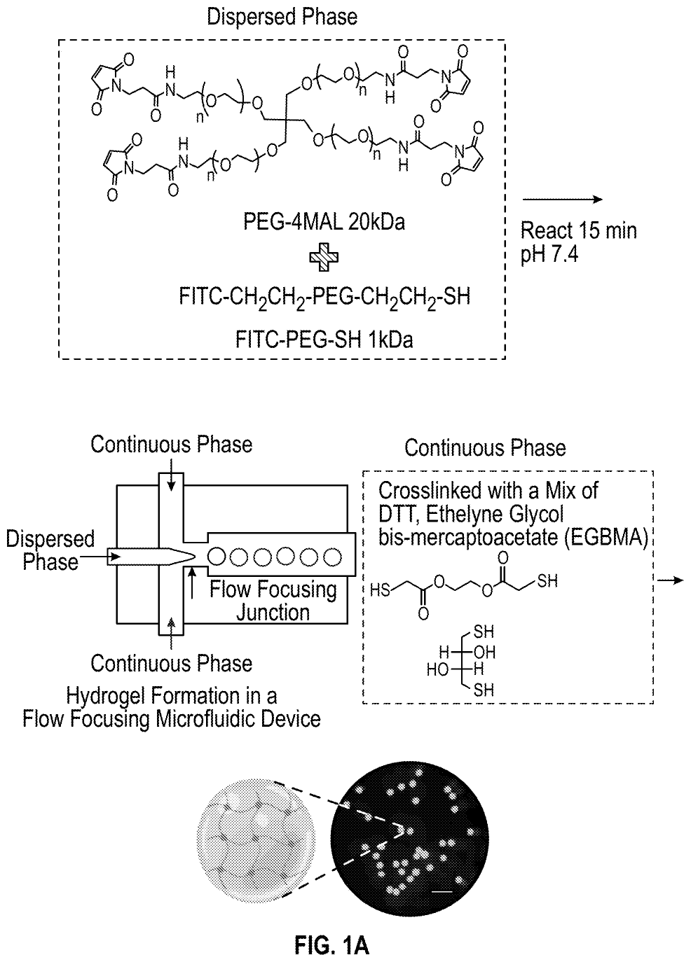

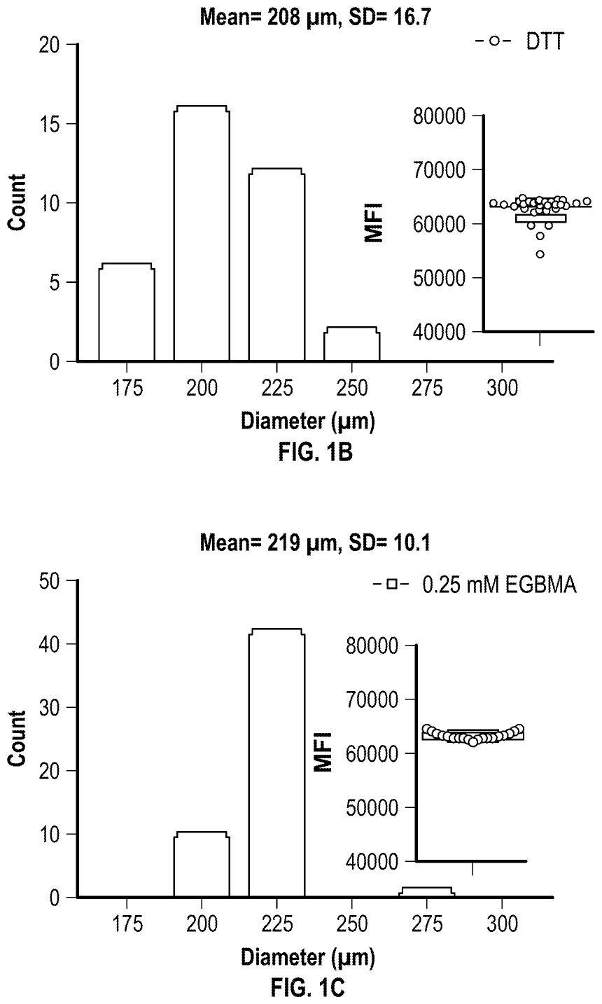

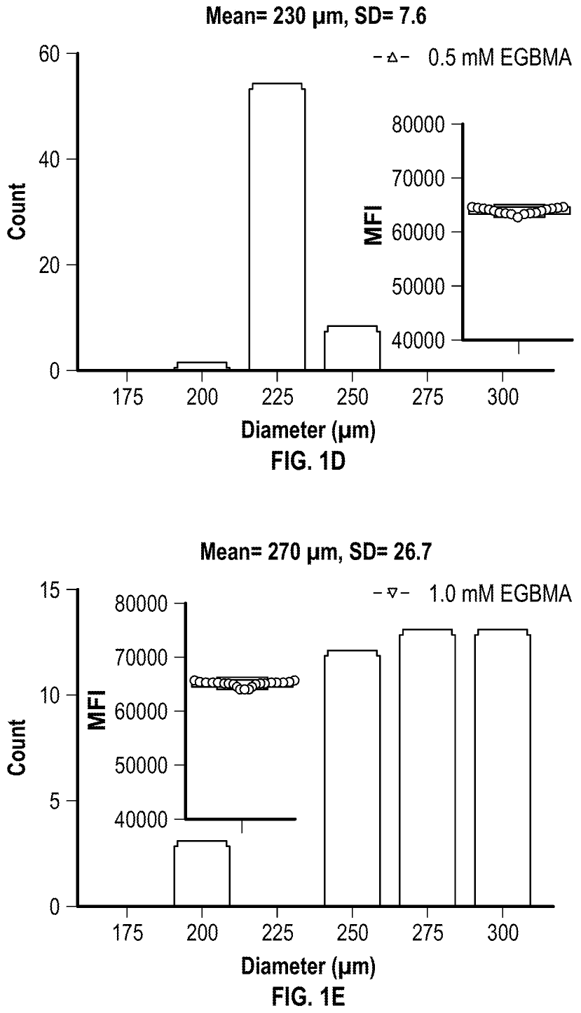

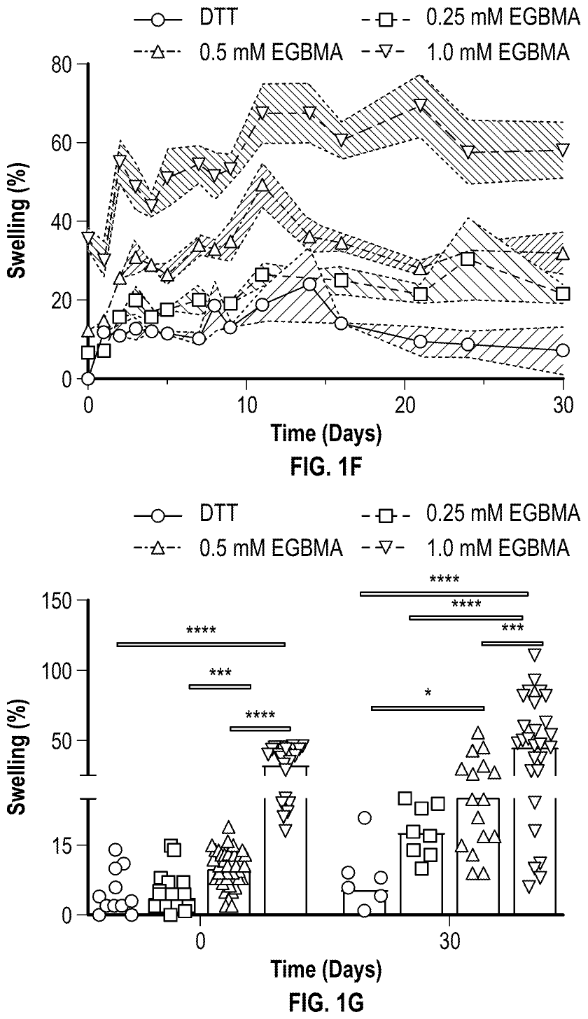

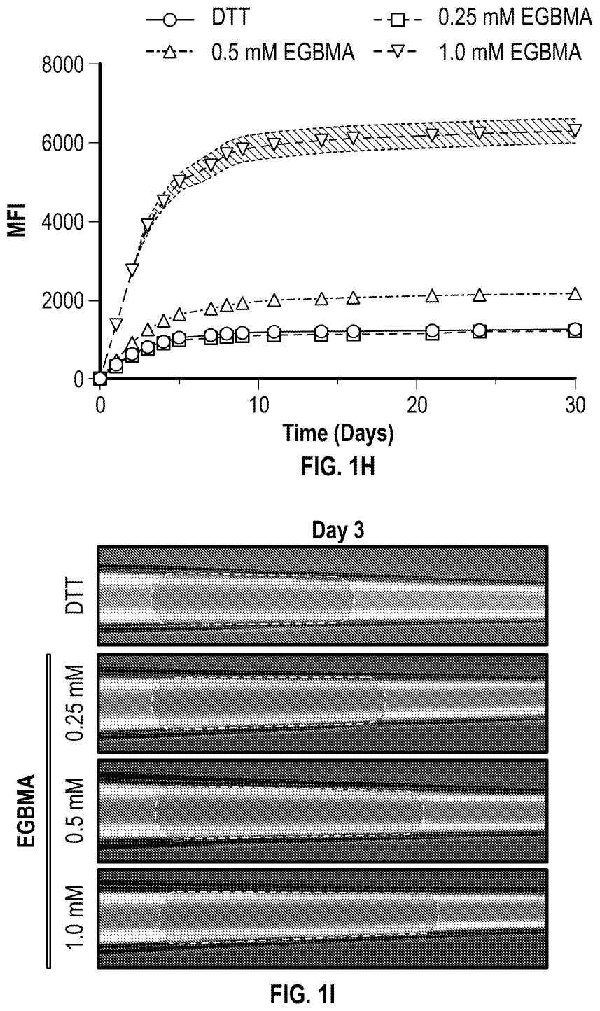

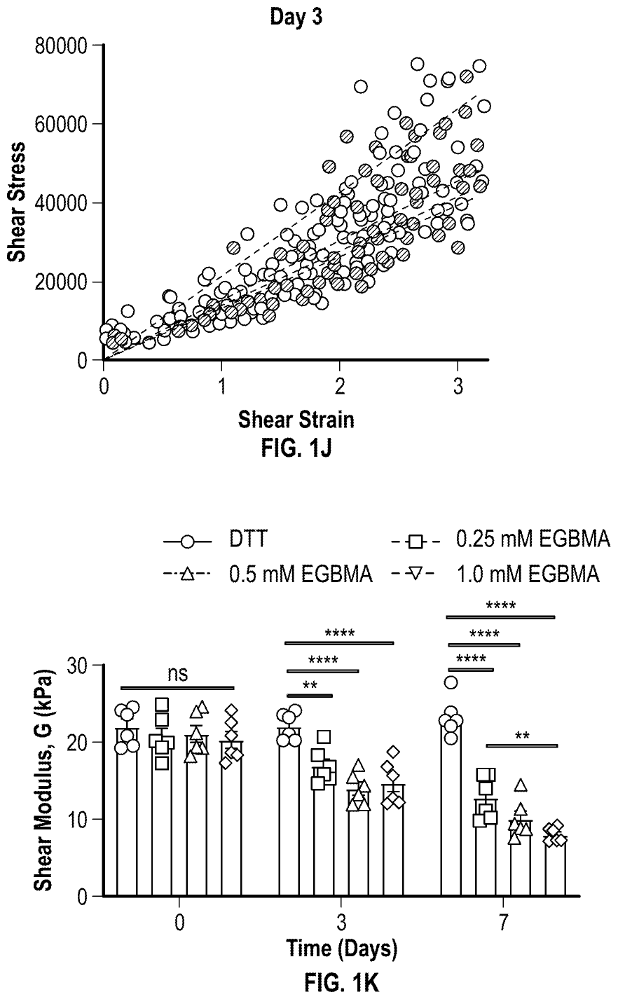

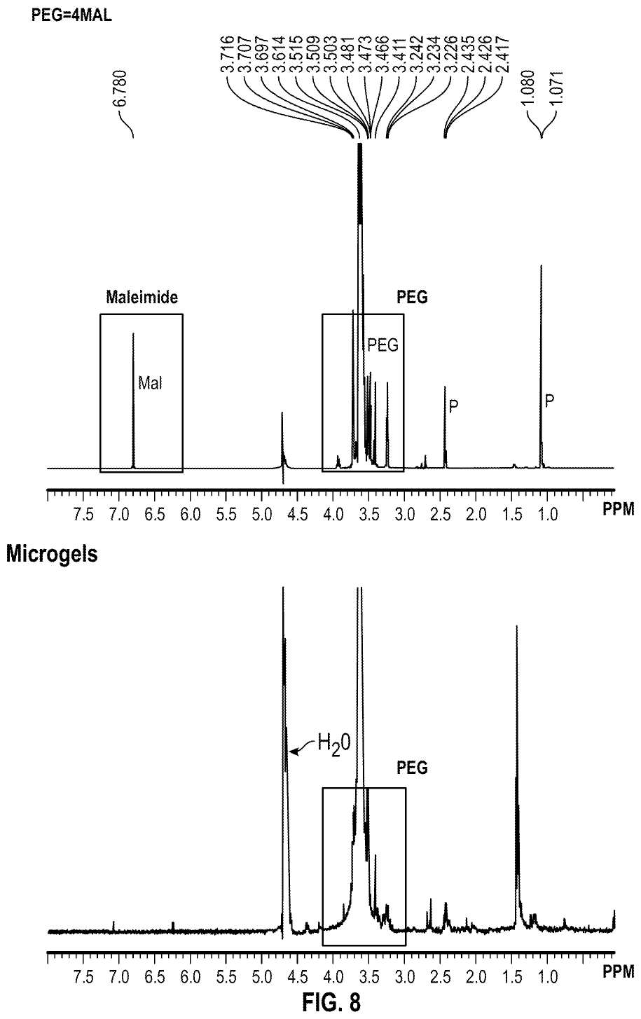

Hydrolytically degradable microparticles (i.e., microgels) were fabricated by droplet segmentation using a flow-focusing microfluidic device, as previously reported (Headen, D. M.; Aubry, G.; Lu, H.; García, A. J. Microfluidic-Based Generation of Size-Controlled, Biofunctionalized Synthetic Polymer Microgels for Cell Encapsulation. Advanced Materials 2014, 26 (19), 3003-3008). The PEG-4MAL macromer was functionalized with a linear PEG-FITC via Michael type addition for particle tracking, prior to segmentation in the microfluidic device. Pumping of the aqueous phase (containing the functionalized macromer) into the microfluidic device-generated droplets that were subsequently covalently crosslinked with a continuous phase of oil containing small dithiol molecules: dithiothreitol (DTT) and ethylene glycol bis(mercaptoacetate) (EGBMA). The addition of EGBMA allowed for the incorporation of a hydrolytically labile ester linker (FIG. 1A and Table 1). The concentration of EGBMA was varied in the continuous crosslinking phase from 0.25, 0.5 to 1.0 mM and held the concentration of DTT constant at 15 mM. ¹H molecular diffusion nuclear magnetic resonance (NMR) of the microgels revealed the absence of maleimide groups in the crosslinked PEG-4MAL macromer, indicating that the maleimide groups in the microgel droplet are efficiently reacted after on chip crosslinking (FIG. 8).

TABLE 1
Microgel samples synthesized

| | Milimolar ratio | | Ethelyne glycol |
|---|---|---|---|
| Sample | of DTT to ethelyne | DTT | bis-mercaptoacetate |
| name | glycol bis-mercaptoacetate | content (mg) | content (mg) |
| DTT | 15:0 | 11.85 | 0.0 |
| 0.25 | 15:0.25 | 11.85 | 0.26 |
| 0.5 | 15:0.5 | 11.85 | 0.52 |
| 1.0 | 15:1.0 | 11.85 | 1.0 |

Maintaining the flow rates constant for all conditions for a device with a 200 μm nozzle led to monodisperse microgel particles averaging 208 μm (CV 8%) in diameter when only implementing DTT in the crosslinking phase (FIG. 1B). Addition of EGBMA to the crosslinking solution generated monodisperse microgel populations (FIGS. 1C-1E, CV <10% for all groups), with microgels ranging in size from 219 to 270 μm diameter. EGBMA crosslinked microgels were 4, 10, and 33% larger in diameter at 4 hr post-fabrication than DTT only microgels, with significant swelling in the highest concentration EGBMA group when compared to the DTT control (FIGS. 1F and 1G, p<0.0001 DTT vs 1.0 mM EGBMA). Macromer functionalization with PEG-FITC was equivalent across groups as seen by mean fluorescent intensity measurements of the microgels post-fabrication (FIGS. 1B-1E), indicating that differences in swelling can be attributed to the presence of the EGBMA linker and not the availability of the maleimide group for crosslinking. By day 30, DTT crosslinked microgels had reached an equilibrium size which was 6% higher than the initial microgel size, whereas EGBMA/DTT microgels with the medium and highest concentration of the labile crosslinker had swollen 26% and 46% greater than their initial sizes, respectively (FIG. 1G, p=0.01 DTT vs 0.5 mM EGBMA, p<0.001 DTT vs 1.0 mM EGBMA). Microgel degradation was also assessed by tracking the amount of PEG-FITC released into solution, as the PEG-FITC is covalently linked to the PEG-4MAL macromer and can only be released from the hydrogel network by hydrolysis of EGBMA. PEG-FITC release results agree with swelling experiments, whereby the 1.0 mM EGBMA crosslinked microgels released PEG-FITC at a faster rate than the lower EGBMA concentrations, while the fully nondegradable control followed a small release of trapped PEG FITC, tailed by no PEG-FITC present in solution as expected.

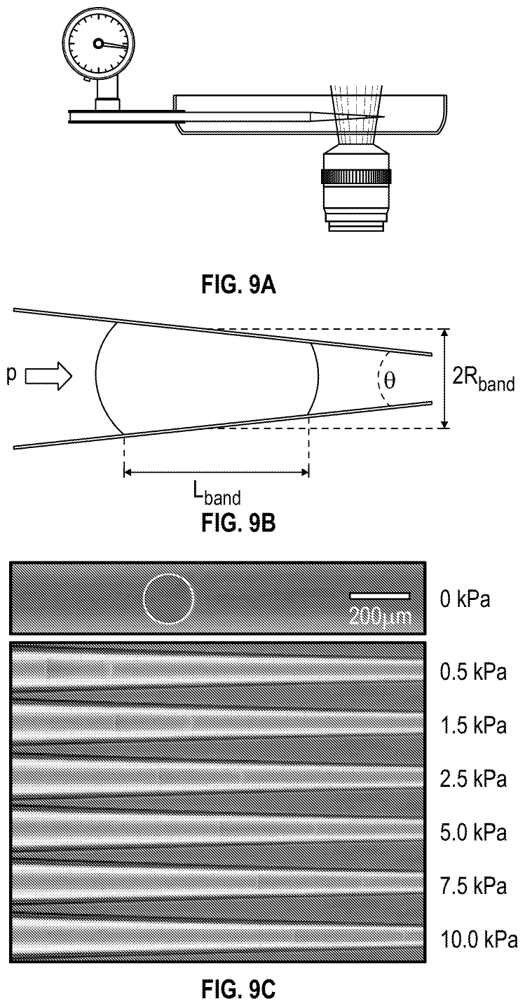

Lastly, the effect of EGBMA on the mechanical properties of the resulting microgels was determined via pressure-induced deformation through a tapered microcapillary (Wyss, H. M.; Franke, T.; Mele, E.; Weitz, D. A. Capillary Micromechanics: Measuring the Elasticity of Microscopic Soft Objects. Soft Matter 2010, 6 (18), 4550-4555) (FIGS. 9A-9C). The microgel is deformed by a pressure differential across the microgel lodged at the end of the microcapillary. As the pressure differential increases, the microgel undergoes radial compressive strain and axial elongation (FIG. 1I). The shear stress and strain can be determined using the taper angle, edge contact length, and average diameter when the microgel is at equilibrium (FIG. 1J). Calculation of shear modulus, G, in this equilibrium state demonstrated no differences in elasticity of the microgels after 4 hr post-fabrication, with values ranging from 20-22 kPa for all groups tested. After 72 hr in solution, shear modulus decreased with increasing EGBMA concentration in the microgels, with a reduction in moduli from 20 kPa to 14 kPa (28% reduction) in the highest degradable linker group (FIG. 1K, p<0.0001 vs DTT); by day 7, elastic modulus had decreased by 61% (8 kPa) in the highest degradable linker group, a behavior explained by reduced crosslink density due to hydrolysis of ester linker. Collectively, these data demonstrate no differences in effective crosslink density immediately following fabrication, while elastic properties of the microgels exhibit time-dependent decreases based on EGBMA hydrolysis and loss of network crosslinks. Furthermore, the results provide evidence of the consistency of the flow-focusing microfluidic platform in fabricating physically and mechanically homogenous microgels.

Microgel Degradation and by-Products do not Induce Monocyte Activation In Vitro

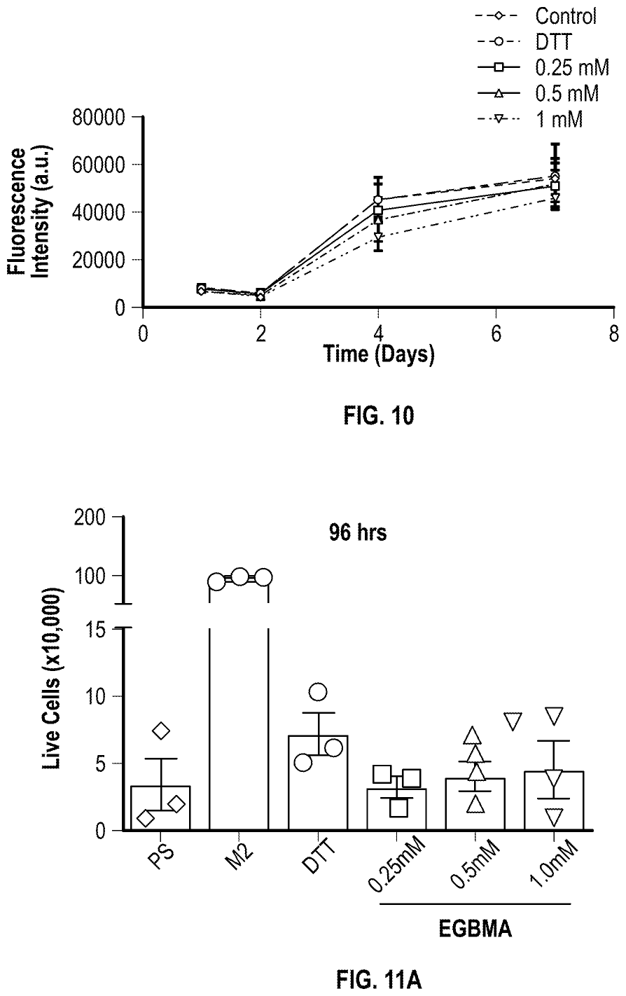

To assess the effects of hydrolytic degradation products of EGBMA/DTT-crosslinked hydrogels on cell viability and activation, the RAW 264.7 mouse macrophages cell line was grown in the presence of the different microgel formulations for over seven days. The presence of the microgels, and fabrication byproducts (e.g. any encapsulated DTT, or byproducts of hydrolysis) were not toxic to this cell line (FIG. 10). These results are consistent with previous work demonstrating no toxicity related to DTT crosslinking for encapsulated cells or cells co-culture with fully crosslinked microgels (Headen, D. M.; Aubry, G.; Lu, H.; García, A. J. Microfluidic-Based Generation of Size-Controlled, Biofunctionalized Synthetic Polymer Microgels for Cell Encapsulation. Advanced Materials 2014, 26 (19), 3003-3008, Coronel, M. M.; Martin, K. E.; Hunckler, M. D.; Barber, G.; O'Neill, E. B.; Medina, J. D.; Opri, E.; McClain, C. A.; Batra, L.; Weaver, J. D.; Lim, H. S.; Qiu, P.; Botchwey, E. A.; Yolcu, E. S.; Shirwan, H.; García, A. J. Immunotherapy via PD-L1-Presenting Biomaterials Leads to Long-Term Islet Graft Survival. Science Advances 2020, and Headen, D. M.; Woodward, K. B.; Coronel, M. M.; Shrestha, P.; Weaver, J. D.; Zhao, H.; Tan, M.; Hunckler, M. D.; Bowen, W. S.; Johnson, C. T.; Shea, L.; Yolcu, E. S.; García, A. J.; Shirwan, H. Local Immunomodulation with Fas Ligand-Engineered Biomaterials Achieves Allogeneic Islet Graft Acceptance. Nature Materials 2018, 17 (8), 732-739).

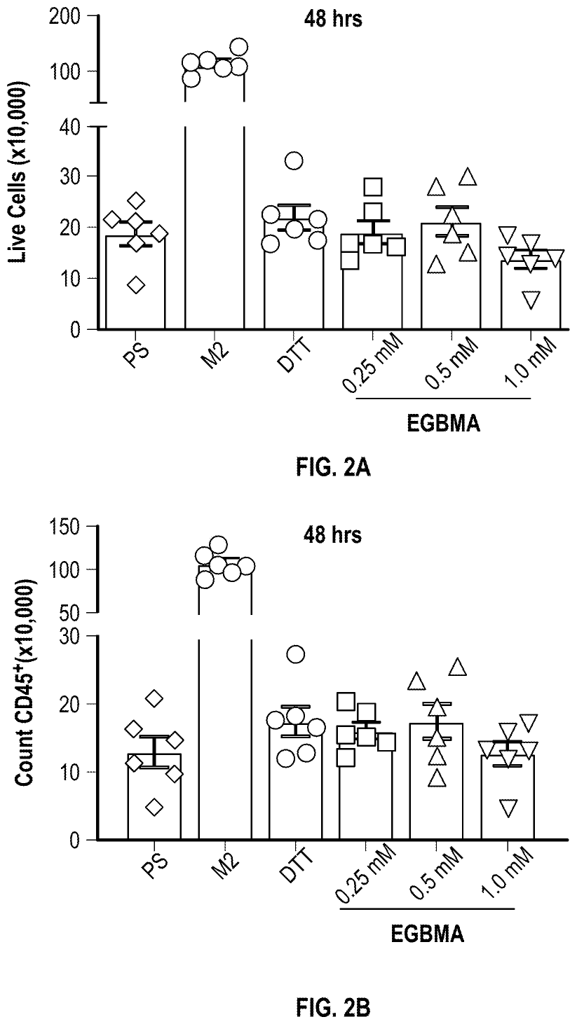

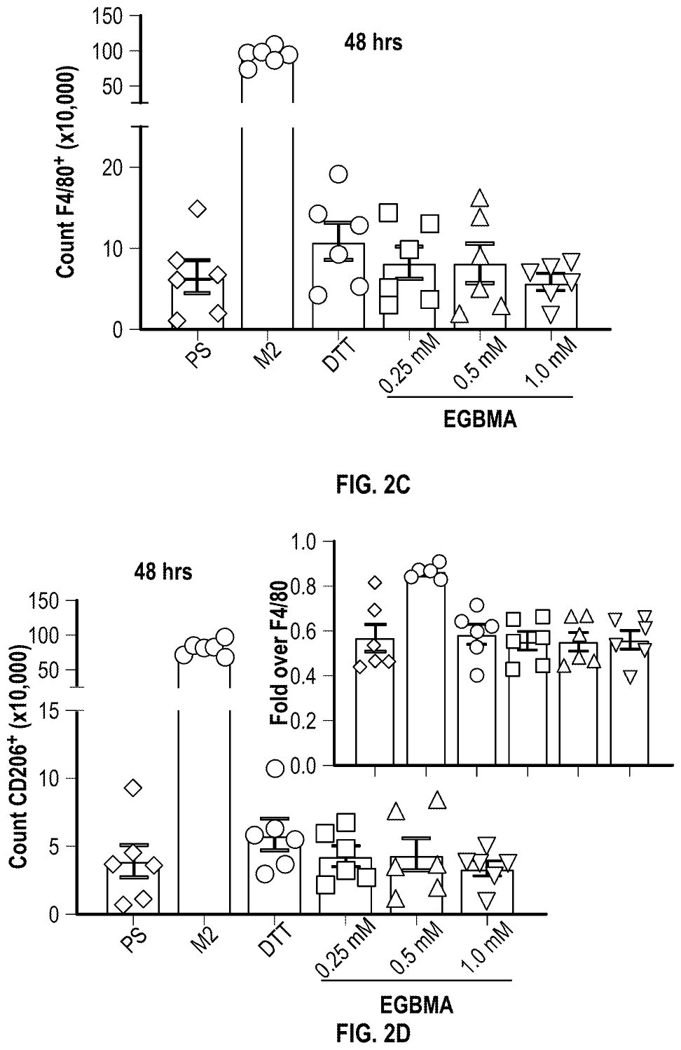

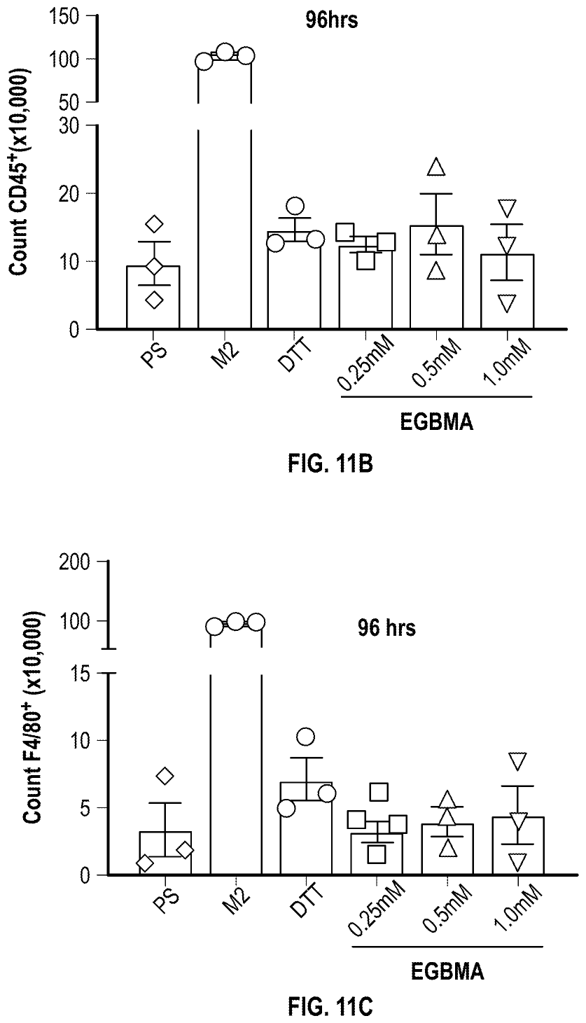

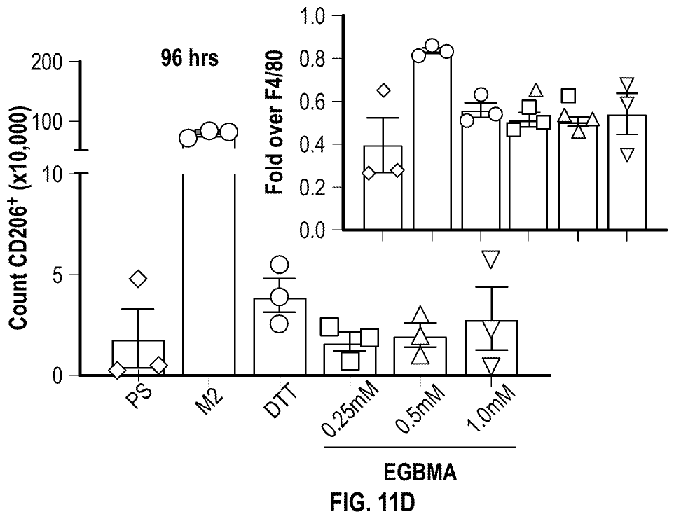

To assess the effects of microgels on immune cell polarization in vitro, a co-culture system was setup involving primary monocytes derived from the bone marrow of C57BL/6J mice with different formulations of crosslinked microgels. Polystyrene beads (PS) of similar size (200 μm) and at the same concentration per well were included as a negative control, as they have been shown to not induce cellular activation (Moore, M. W.; Cruz, A. R.; LaVake, C. J.; Marzo, A. L.; Eggers, C. H.; Salazar, J. C.; Radolf, J. D. Phagocytosis of Borrelia Burgdorferi and Treponema Pallidum Potentiates Innate Immune Activation and Induces Gamma Interferon Production. Infection and Immunity 2007, 75 (4), 2046-2062). Furthermore, an IL-4-polarized M2 regulatory phenotype was included as a positive control, as exposure of macrophages to bulk PEG hydrogels has been shown to shift cell polarization towards a regulatory phenotype in the absence of adhesion cues and inflammatory signals (Lynn, A. D.; Bryant, S. J. Phenotypic Changes in Bone Marrow Derived Murine Macrophages Cultured on PEG-Based Hydrogels and Activated by Lipopolysaccharide. Acta Biomater 2011, 7 (1), 123-132). Microparticle-containing groups exhibited similar cell viability following 48 hr of co-culture with all microgel formulations or PS (FIG. 2A), while the addition of IL-4 led to an increase in cell numbers in the co-culture. No changes in the expression of CD45, F4/80, and regulatory marker CD206 were observed in the presence of PS or PEG-based microgels after two days of co-culture (FIGS. 2B-2D). In addition, the overall expression of these markers was equivalent after 4 days of co-culture (FIGS. 11A-D). These findings demonstrate that changes in microgel elastic properties and size or degradation products do not induce any phenotypic changes in macrophage marker expression under noninflammatory conditions in vitro.

### Subcutaneous Microgel Implantation Leads to Controlled Degradation In Vivo

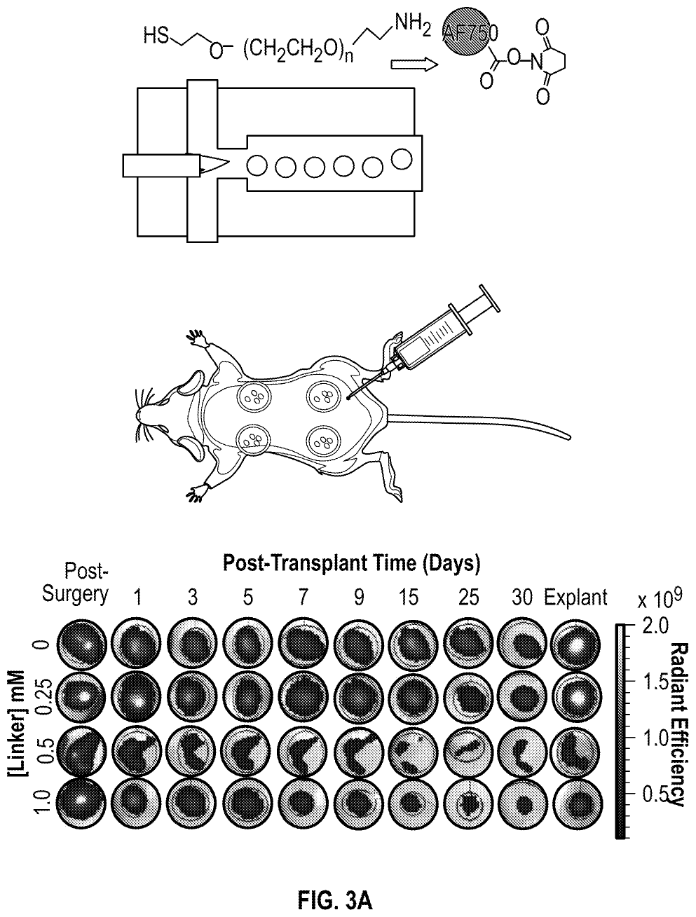

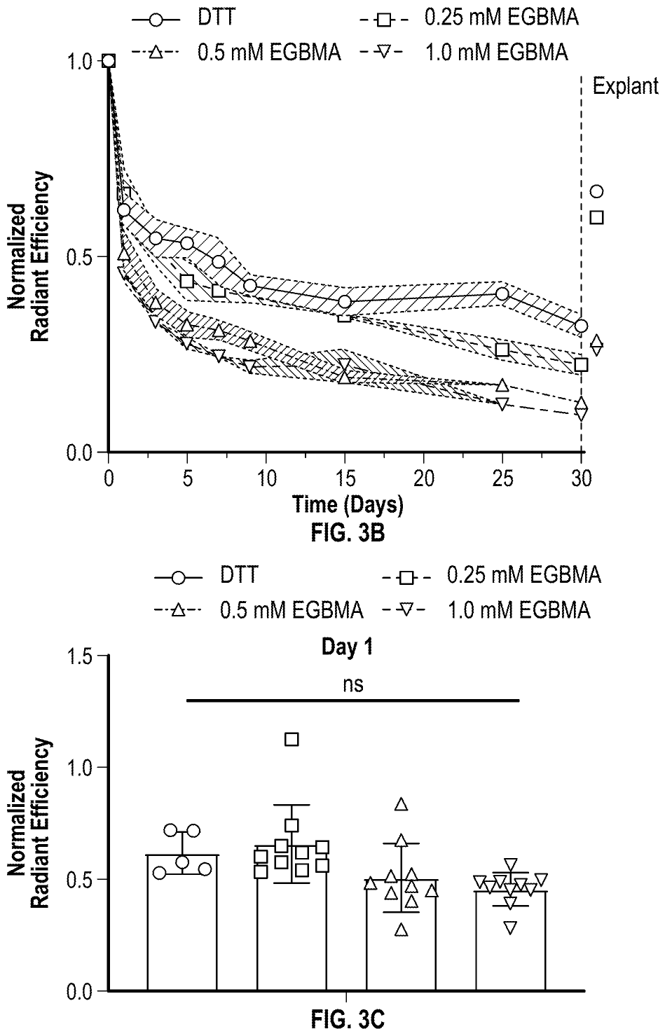

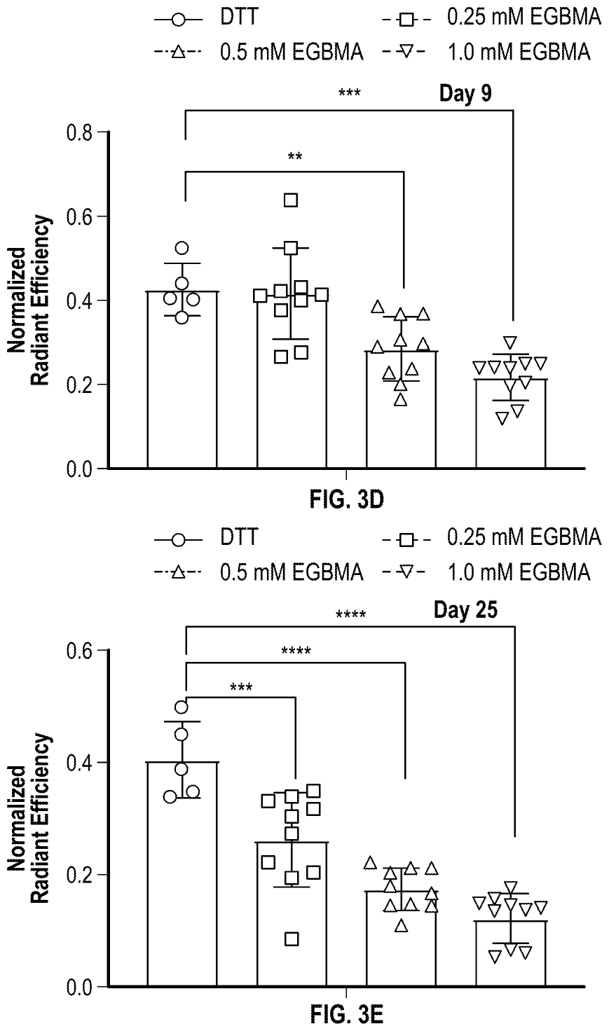

To test the ability of EGBMA/DTT-crosslinked hydrogels to be degraded in vivo, microgels were fabricated as described above but the PEG-FITC tracker was replaced by a linear PEG of the same molecular weight containing a near-infrared dye for in vivo tracking. Microgels were injected into subcutaneous pockets in the dorsum of albino mice (to avoid attenuation of signal detection by melanin pigmentation (Curtis, A.; Calabro, K.; Galarneau, J.-R.; Bigio, I. J.; Krucker, T. Temporal Variations of Skin Pigmentation in C57Bl/6 Mice Affect Optical Bioluminescence Quantitation. Mol Imaging Biol 2011, 13 (6), 1114-1123)). The degradation of the microgels was tracked via in vivo fluorescent imaging (IVIS) (FIG. 3A). Normalized radiant efficiency tracking over time demonstrates a decrease in fluorescence signal that is dependent on EGBMA concentration (FIG. 3B). Notably, while there was a decrease in signal intensity in the DTT-crosslinked group, intensity values post-explant were comparable to day 1 values, demonstrating no degradation in this group as expected (FIG. 3B, post-explant values after dashed lines, p=0.44). No differences in fluorescence signal among microgel formulations were observed on day 1 post-injection (FIG. 3C), yet by day 9, fluorescence signal was significantly lower in microgel formulations containing the intermediate and highest concentrations of EGBMA crosslinker (FIG. 3D, p=0.008, p=0.0001 vs DTT respectively). On day 25, signal intensity was 26%, 17%, and 12% of the original signal, a decrease directly proportional to the EGBMA linker concentration (FIG. 3E, p=0.0004, p<0.0001, p<0.0001 vs DTT). Thus, IVIS imaging confirmed that microgels crosslinked with EGBMA degrade in vivo, and this degradation occurs over several weeks.

### Degradation Properties Regulate Immune Response to Microgels In Vivo

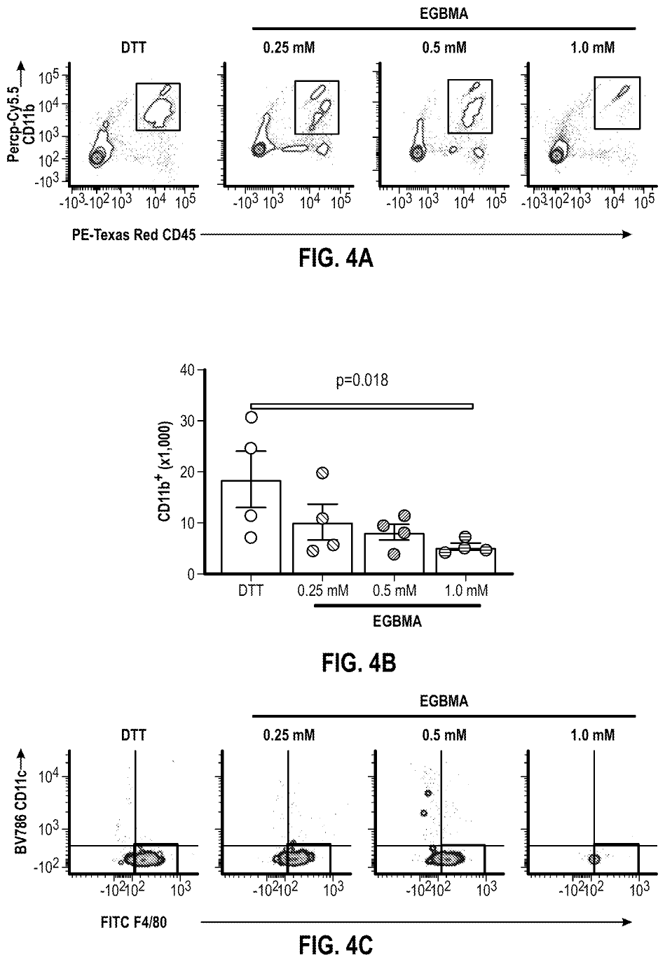

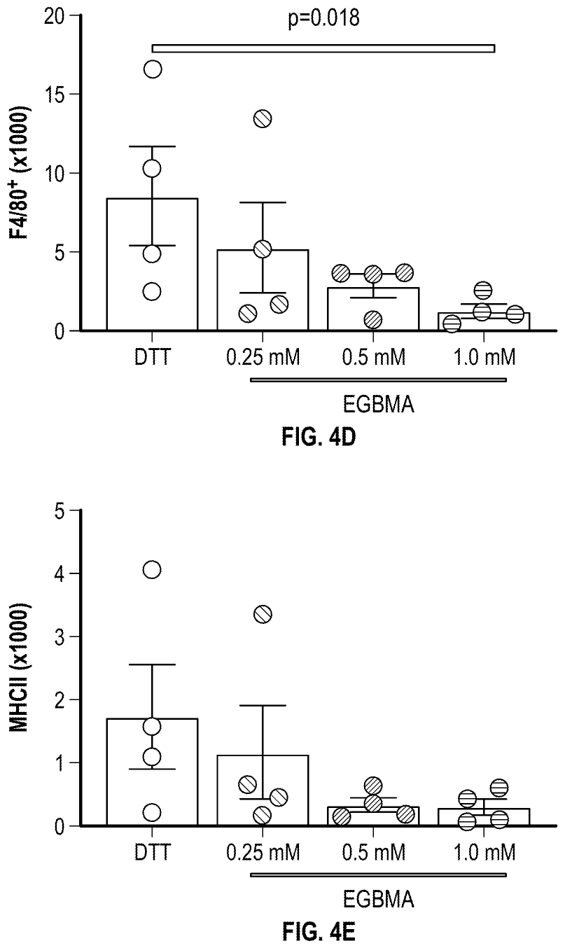

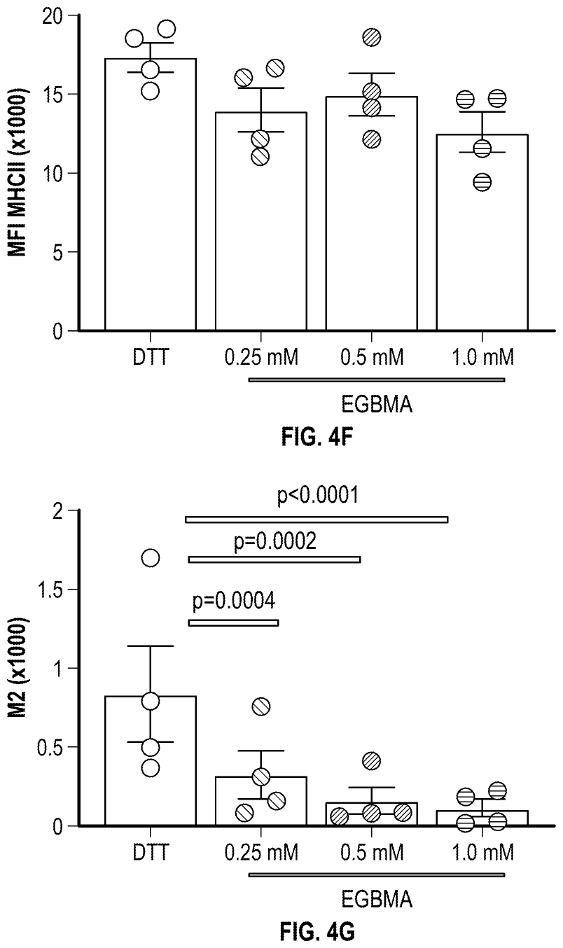

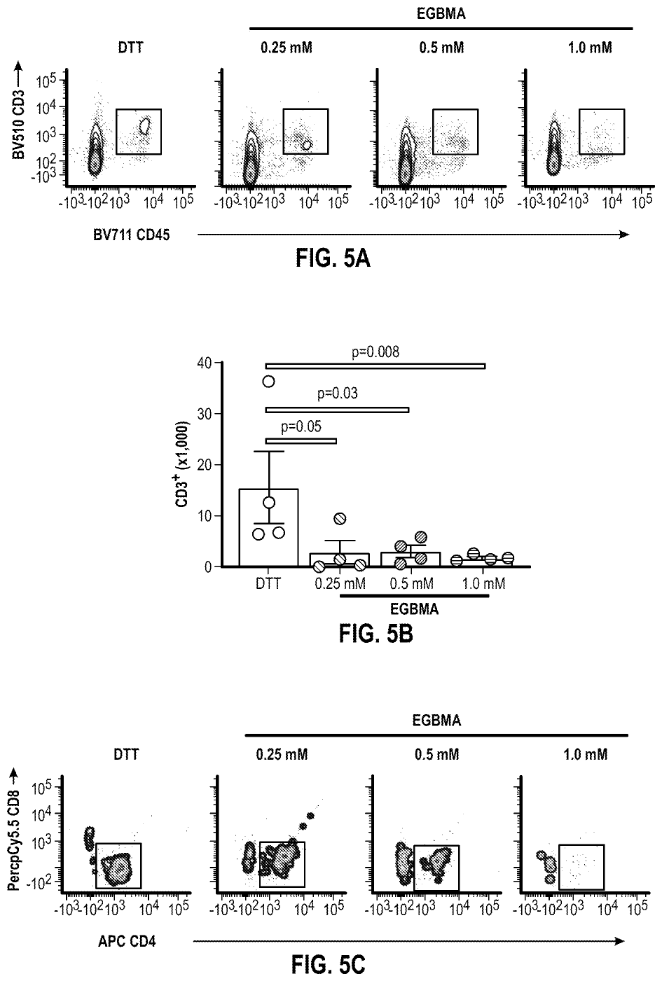

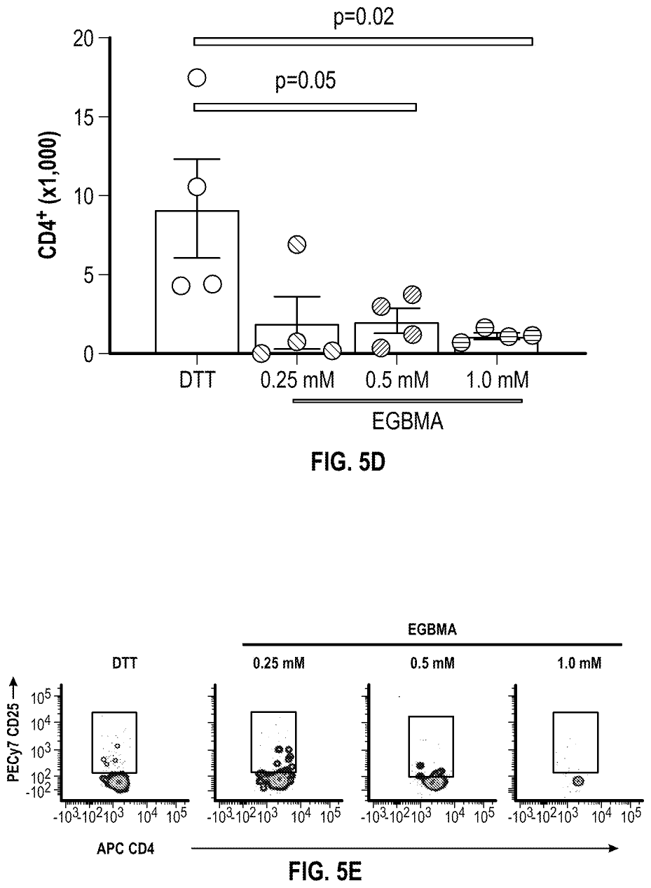

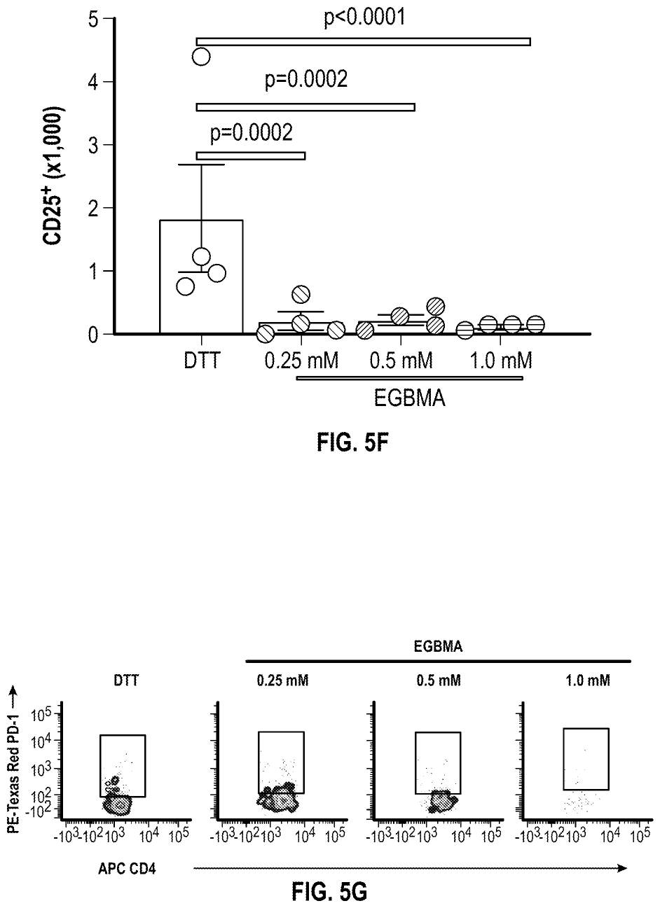

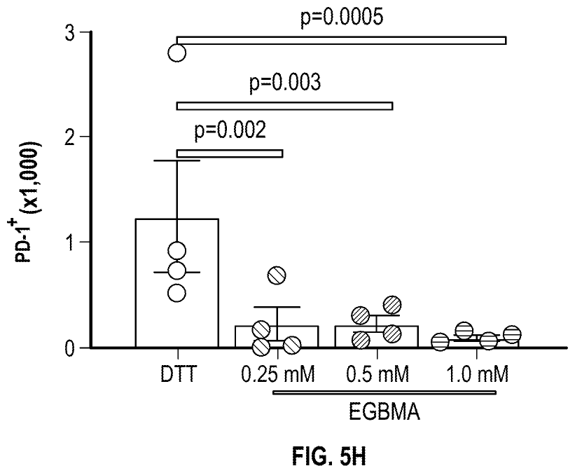

It was hypothesized that degradation and changes in the mechanical properties of the microgels due to swelling and hydrolysis of the ester-containing linker would influence immune responses to the implants. All four different microgel formulations were injected into subcutaneous dorsal pockets of BALB/cJ mice and retrieved the tissue on day 7 for multiparametric flow analysis. This time point was chosen as it had demonstrated differences in mechanical properties and degradation profiles among the materials being tested. Furthermore, to assess how degradability and dynamic changes in mechanical properties influence the cellular environment, the analysis focused on comparisons between non-degradable (DTT) and the three different degradable formulations. Myeloid cell populations (CD45+CD11b+) were dominant at the injection site in the microgels crosslinked with DTT compared to the degradable microgels containing 1.0 mM EGBMA (FIG. 4A-4B, p=0.018). Phenotyping of subpopulations within the myeloid compartment revealed differences in the presence of F4/80+ macrophages as a function of microgel crosslinker formulation (FIG. 4C-4D, p=0.008). Measuring activation of this cell population based on cell expression of major histocompatibility complex class II (MHCII) revealed no differences among microgel formulations (FIG. 4E-4F), yet intensity expression of this marker was greatest in the DTT crosslinked group when compared to the highest degradable microgel formulation (FIG. 4F, p=0.03 DTT vs 1.0 mM EGBMA). Further analysis of macrophage polarization using the M1- and M2-associated markers, CD86 and CD206, indicated an increased presence of M2 polarized F4/80-expressing macrophages in the nondegradable DTT group compared to all other degradable formulations (FIG. 4G), showing that tissue responses to nondegradable nonphagocytable microgels are dominated at this time point by an M2-like phenotype, and this polarization can be regulated by the material degradation profile. The effect of microgel formulation was examined next on the recruitment of T cells to the injection site. DTT-crosslinked microgels recruited a higher number of CD3+ cells to the implant pocket compared to degradable microgels (FIG. 5A-5B). This lymphocytic response was dominated by CD4 helper cells, which were elevated in the presence of nondegradable microgels (FIG. 5C-5D). Expression of activation markers CD25 and PD-1 (FIGS. 5E-5H) was also influenced by microgel formulation, with increased upregulation in surface expression of these markers observed in the nondegradable group compared to the degradable microgels. Overall, the enhanced presence of CD4 cells combined with an increased M2-like cell phenotype in the DTT group, demonstrates an interplay between these two cell populations in the tissue response to nondegradable PEG-based microgels. Moreover, microgel degradation profiles, including changes in mechanical properties, can modulate the recruitment and phenotype of specialized cell subpopulations altering host tissue responses to the biomaterial implant.

### Microgel-Induced Cytokine Milieu is Dynamic and Dominated by IFN-γ Expression

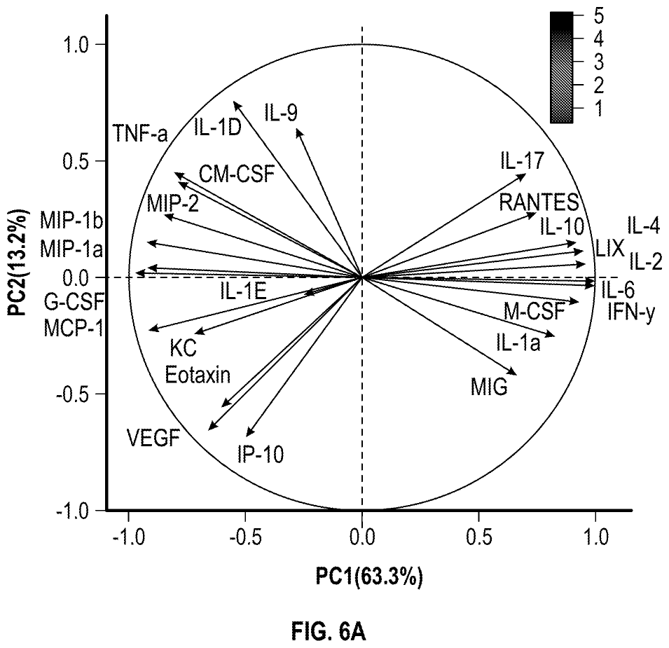

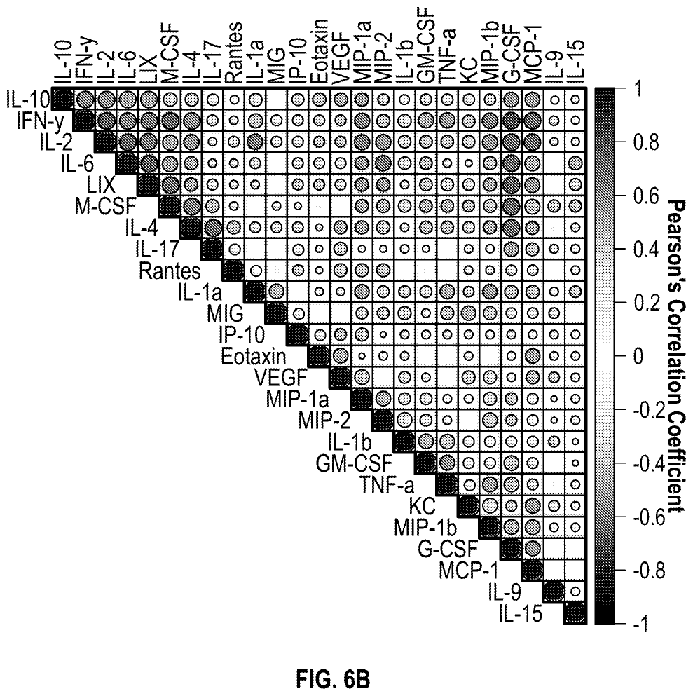

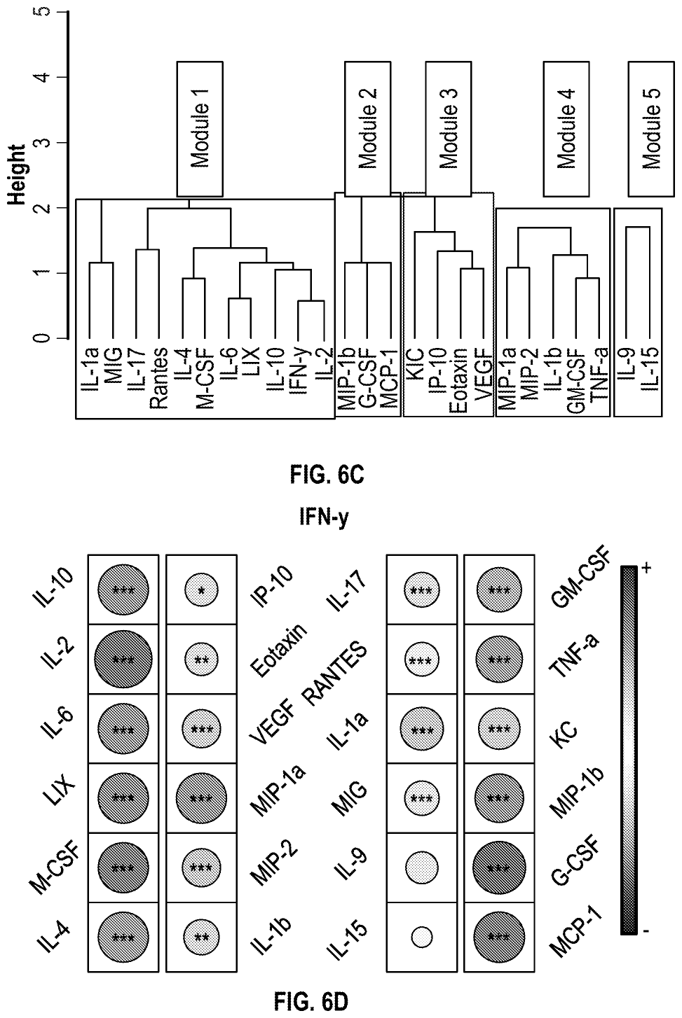

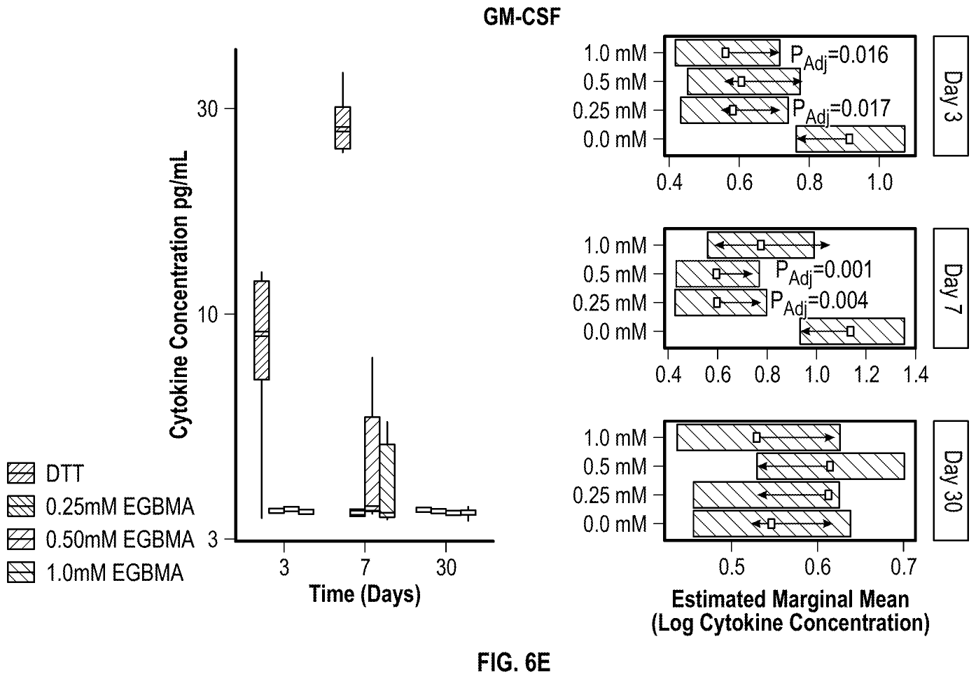

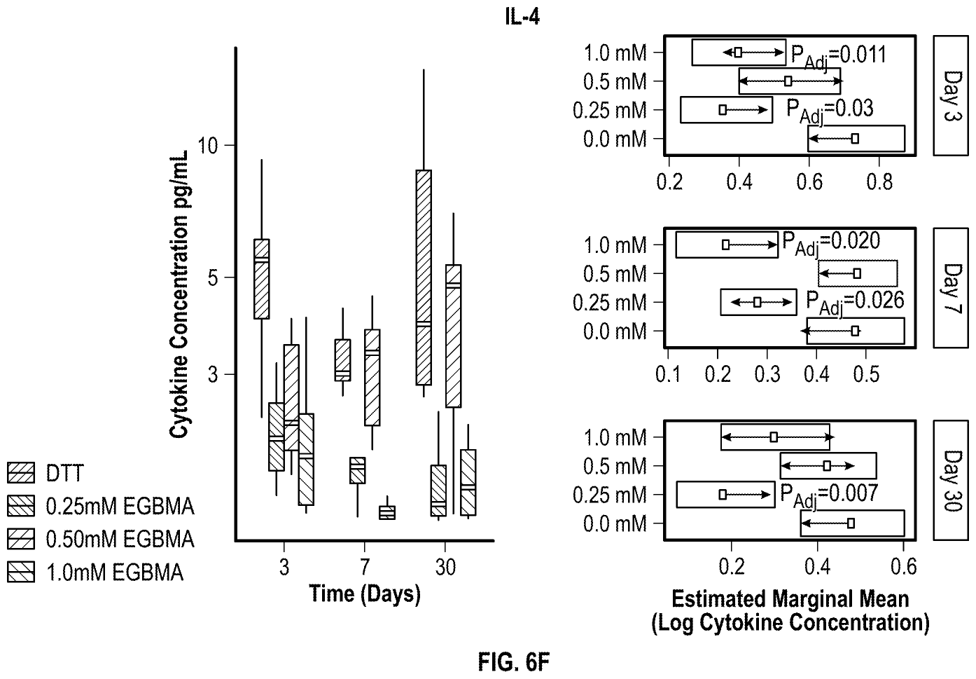

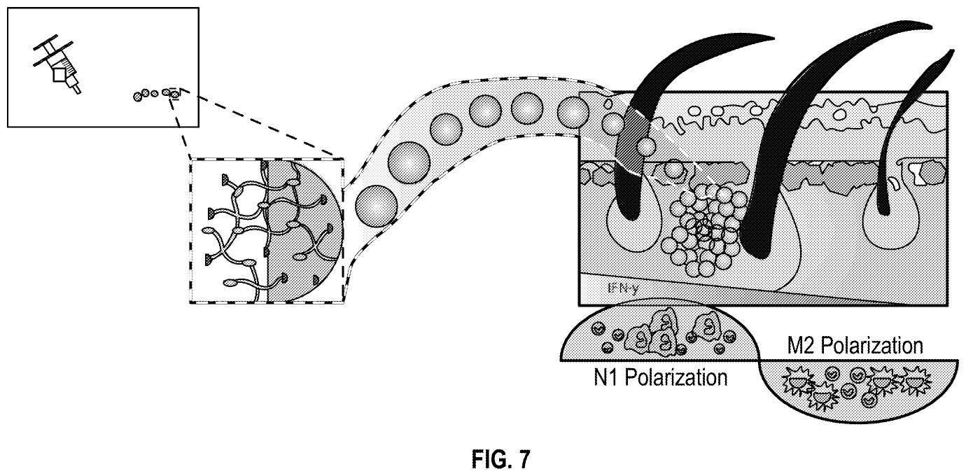

To better understand the interplay of the immune environment with the microgel degradation profile, a multiplexing technique was implemented to investigate the cytokine and chemokine (hereon referred to as cytokines) milieu regulating T cell recruitment and macrophage polarization post-microgel injection. Additionally, given the high level of correlation between cytokines, and the potential confounding factor of mice age on cytokine release (4-week difference between first and last time point analyzed), a modular cytokine analysis method, CytoMod, was implemented to provide some context between cytokine clustering and the observed cell phenotypes, as opposed to evaluating individual cytokines at distinct time points (Cohen, L.; Fiore-Gartland, A.; Randolph, A. G.; Panoskaltsis-Mortari, A.; Wong, S.-S.; Ralston, J.; Wood, T.; Seeds, R.; Huang, Q. S.; Webby, R. J.; Thomas, P. G.; Hertz, T. A Modular Cytokine Analysis Method Reveals Novel Associations With Clinical Phenotypes and Identifies Sets of Co-Signaling Cytokines Across Influenza Natural Infection Cohorts and Healthy Controls. Frontiers in Immunology 2019, 10, 1338 and Distinct inflammatory profiles distinguish COVID-19 from influenza with limited contributions from cytokine storm https://www.science.org/doi/10.1126/sciadv.abe3024 (accessed 2021-10-25)). Principal component analysis (PCA) of grouped cytokines identified two directions in cytokine profile, with the bulk of the variation in cytokine levels dictated by cytokines IFN-γ and IL-2 in one direction and G-CSF in the orthogonal direction (FIG. 6A, vector direction and color represent the contribution to the PCA). Cytokine similarity across all subjects was defined by their Pearson correlation coefficient (FIG. 6B), whereby unsupervised hierarchical clustering was used to identify five cytokine modules (FIG. 6C). A statistically significant correlation was seen in module 1, composed of cytokines and chemokines involved in inflammation and Th polarization responses (IFN-γ, IL-2, IL-4, IL-17, IL-10, IL-6, MIG, RANTES, M-CSF, LIX). A cytokine-specific score was computed between cytokine levels and the mean cytokine matrix of all subjects, which determined IFN-γ as the driving cytokine consistent with the previous PCA analysis. Correlation plots within this module indicate a statistically significant positive correlation among most of the cytokines (IL-2, IL-10, IL-6, LIX, M-CSF) and IFN-γ. Thus, a condition with a high expression of IFN-γ was relatively likely to display a high concentration of these other cytokines (FIG. 6D).

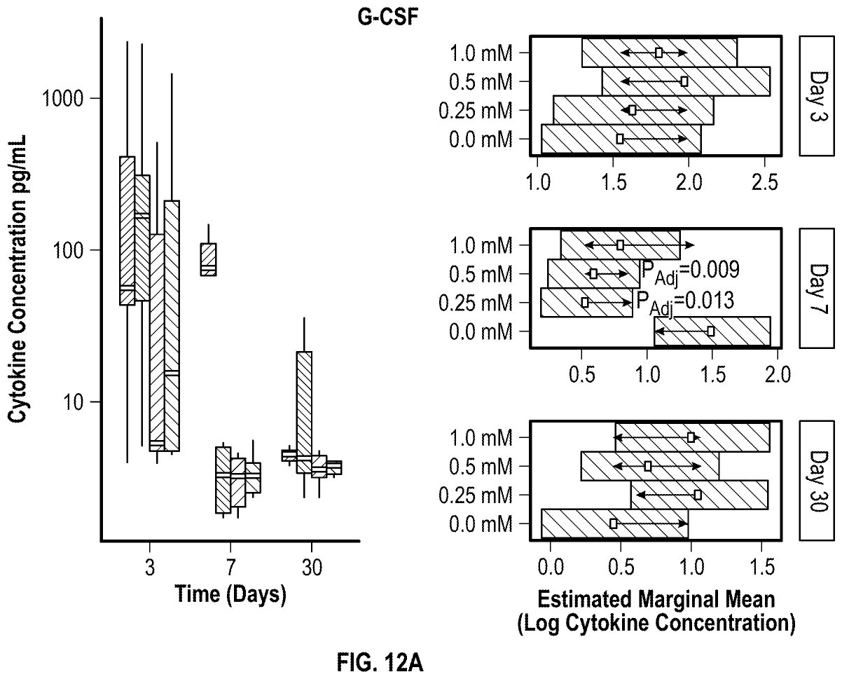

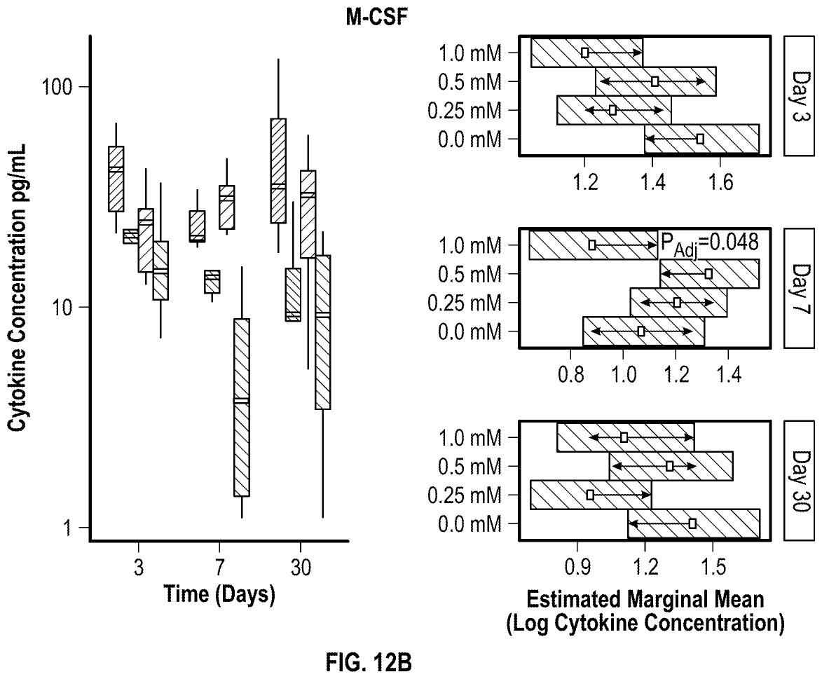

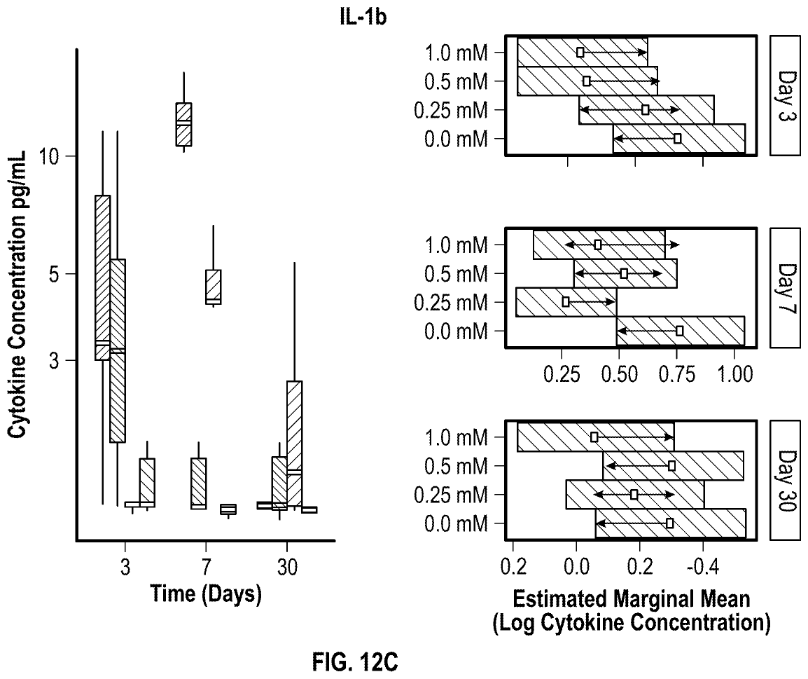

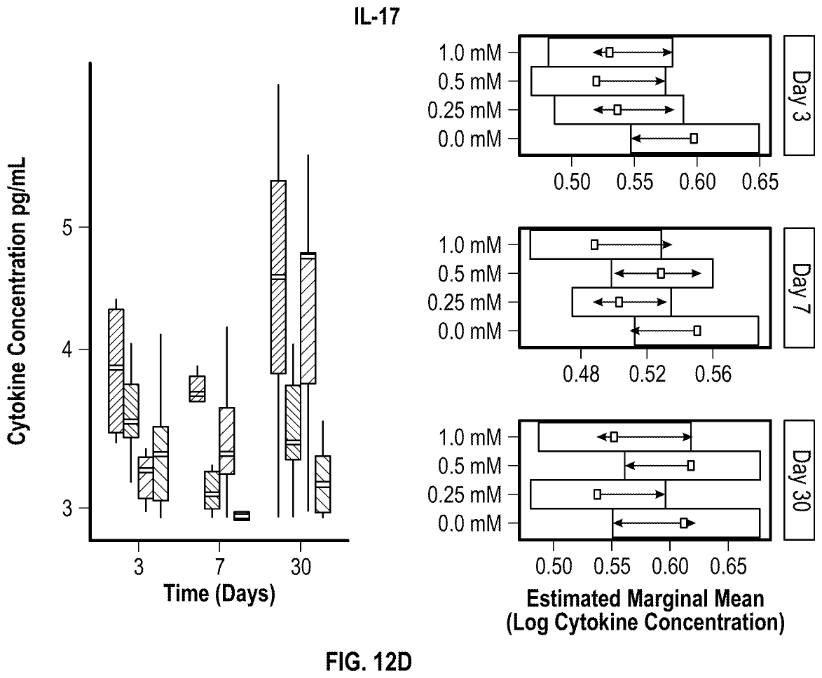

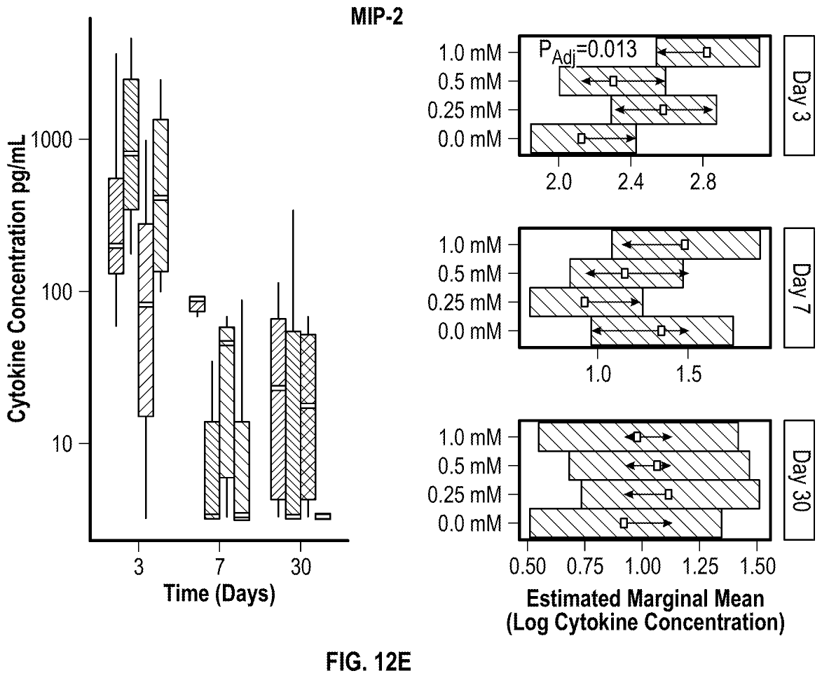

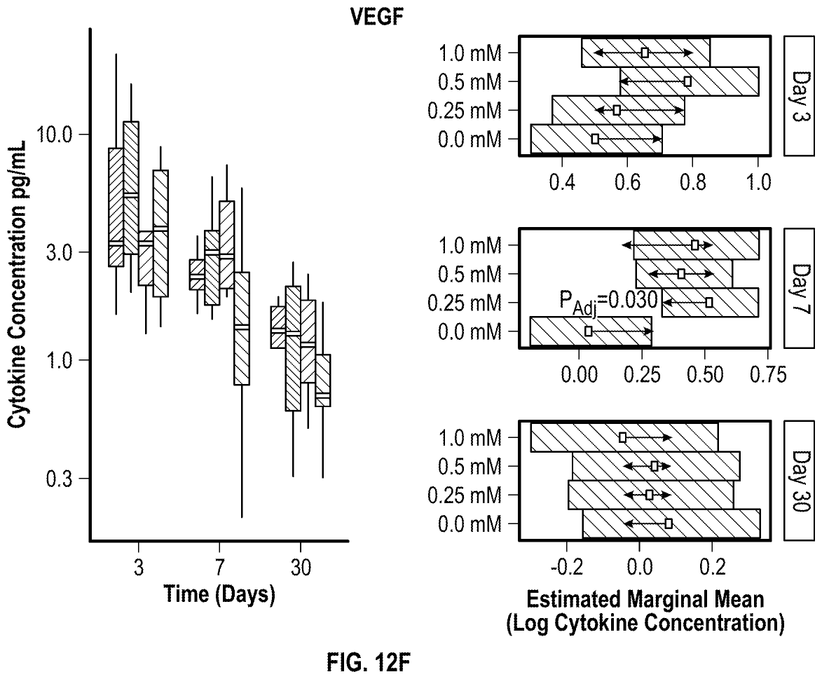

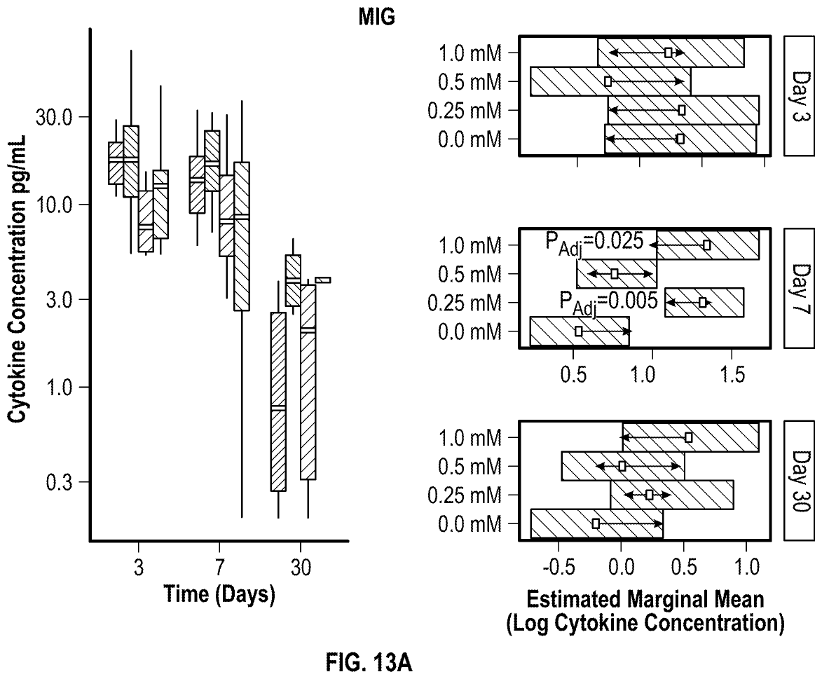

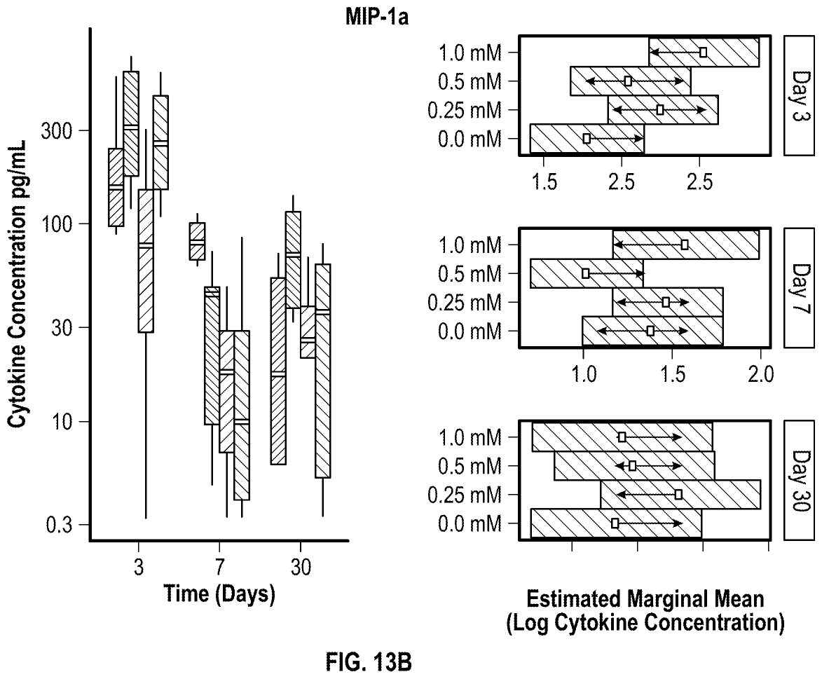

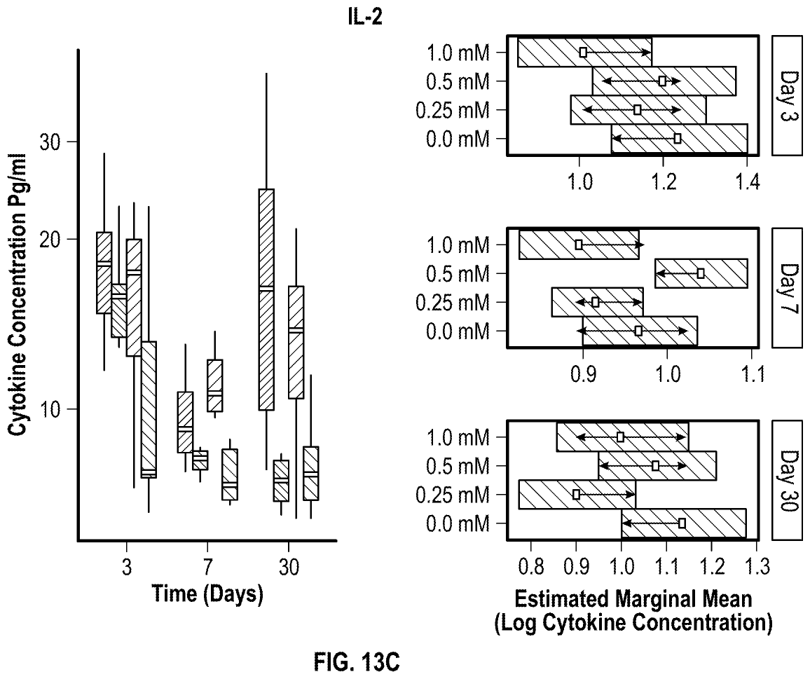

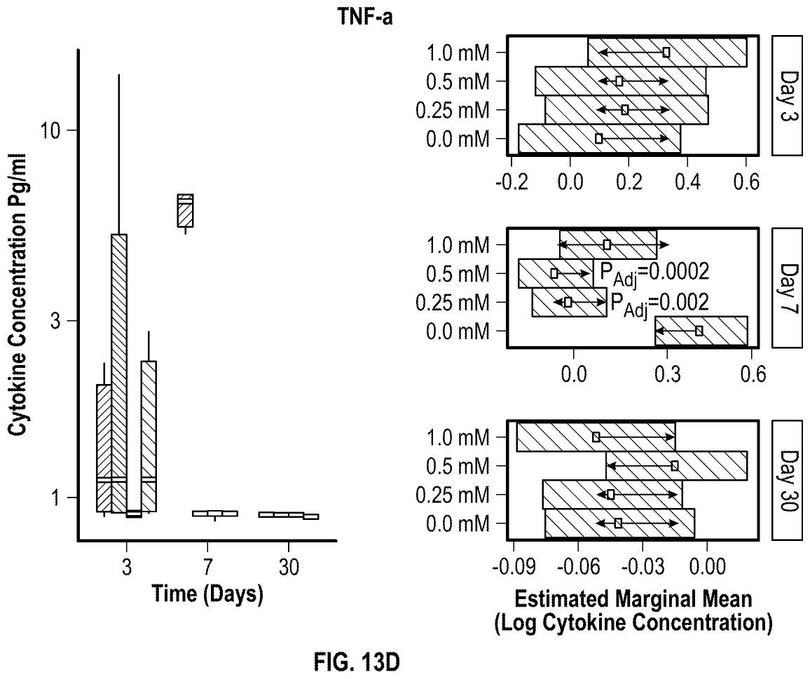

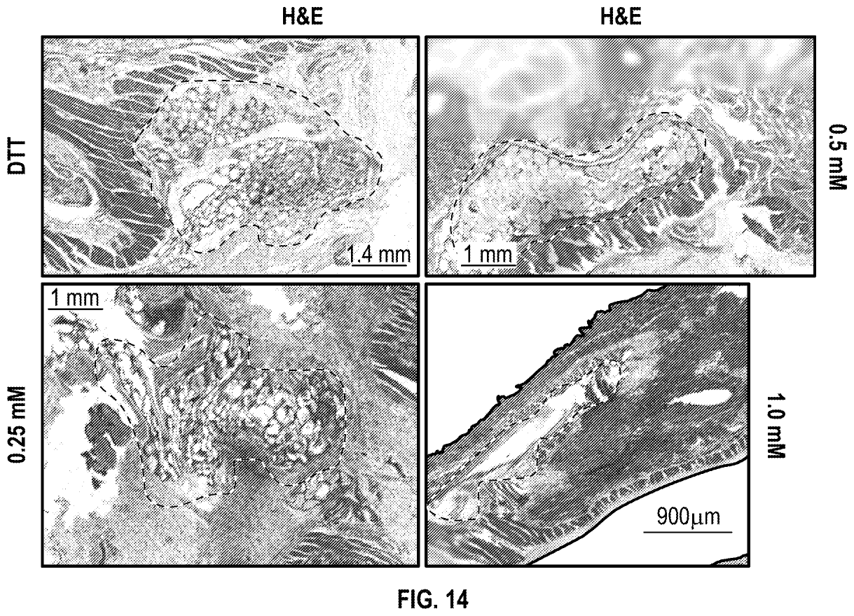

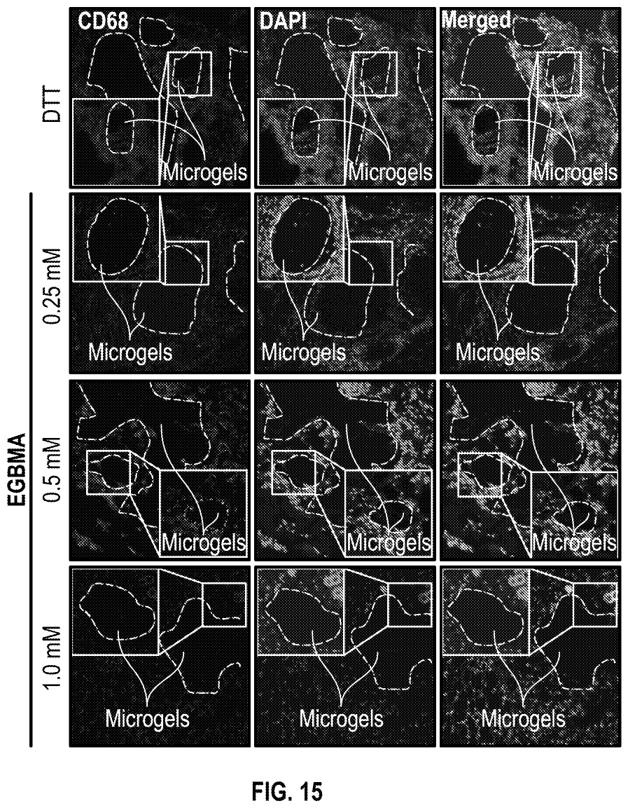

Direct comparison of raw cytokine values for all conditions at all time points evaluated indicated dynamic profiles, with EGBMA-containing formulations presenting lower mean cytokine levels for most cytokines evaluated (FIGS. 6E-6F, FIGS. 12A-12F, FIGS. 13A-13D). Nondegradable microgels resulted in increased expression of GM-CSF and G-CSF early post-injection (FIG. 6E, FIGS. 12A-12F). By day 7, expression of other chemokines involved in immune cell recruitment such as M-CSF and monokine induced by IFN-γ (MIG) was reduced in the group with the highest EGBMA degradable linker compared to the nondegradable control (FIGS. 12A-12F and 13A-13D). Expression of type 1-associated cytokines TNF-α and the IFN-γ driver IL-2 was also reduced in tissues exposed to degradable microgels (FIGS. 12A-12F and 13A-13D). Of note, expression of the crucial type 2 cytokine IL-4 was dependent on the microgel formulation, with higher levels of IL-4 observed at all time points for the nondegradable microgel compared to all degradable formulations (FIG. 6F). Additionally, no differences in expression of angiogenic factor VEGF were observed among formulations at the time points investigated (FIGS. 12A-12F). These results, combined with the multiparametric flow analysis of cell phenotypes at the injection site, indicate that responses to PEG-based nondegradable microgels are driven by type 1 immunity with a degree of cross-regulation by type 2 driving cytokine IL-4. Importantly, this response can be modulated by the introduction of labile ester groups that hydrolytically degrade in vivo. Tissue response to microgel injections in the dorsum of albino mice were generally mild, with cell infiltrates surrounding the implant periphery (FIG. 14). Of note, none of the implants showed signs of encapsulation, or cyst formation. Cellular deposition around the surrounding microgel implant was apparent at the host implant boundary at 4 weeks post-implantation (FIG. 15). Qualitative assessment of images revealed a higher cellular density around the implant periphery of implants containing nondegradable microgels (DTT), compared to all formulations of degradable microgels. Immunohistochemistry for pan macrophage marker CD68 demonstrated and increased presence of CD68 expression in the DTT and 0.25 and 0.5 mM EGBMA conditions compared to the condition containing the highest concentration of EGBMA (1.0 mM EGBMA). These results are consistent with flow cytometry assessments for myeloid populations (FIGS. 4A-4G).

### Discussion and Conclusion

Strategies conferring degradability to microgels have taken advantage of degradable chains in the polymer backbone or labile crosslinker units to enable cleavage either via hydrolysis, enzymatic reaction, or dissolution. Previously, protease degradable microgels were generated for the delivery of angiogenic factors (Foster, G. A.; Headen, D. M.; Gonzalez-García, C.; Salmerón-Sanchez, M.; Shirwan, H.; García, A. J. Protease-Degradable Microgels for Protein Delivery for Vascularization. Biomaterials 2017, 113, 170-175). While these microgels were formed implementing droplet microfluidics, it required the design of a custom microfluidic device, given the crosslinking peptides' limited solubility in the continuous phase. Here, a fabrication strategy is presented that takes advantage of ester hydrolysis to regulate the degradation of crosslinked PEG-4MAL microgels. In contrast to previous approaches, this strategy can be implemented in the same microfluidic device previously designed for the fabrication of nondegradable microgels, as the labile crosslinker unit can be added to the oil crosslinking phase. Thus, this strategy enables tuning of the degradation properties of the microgel product simply by adjusting the crosslinking feed.

Hydrogel degradation was monitored by evaluating changes in physical and mechanical properties, including swelling, release of a PEG-FITC tag, and elastic modulus. Changes in these parameters were directly related to the EGBMA crosslinker content, and thus the number of hydrolyzable groups. Immediately post-fabrication, microgels synthesized with the highest concentration of labile ester junctions swelled to ˜140% of the nondegradable microgel control's size; however, no appreciable differences in elastic modulus were observed at this point. This is explained by the fact that, to completely release the PEG-4MAL macromer, multiple ester bonds must be cleaved. Indeed, measurable changes in elastic modulus were first observed following 72 hr in aqueous buffer, when sufficient crosslinks had been cleaved and PEG-4MAL macromer dissolution into the aqueous medium had occurred. Differences were more pronounced with time and directly proportional to EGBMA content, demonstrating the tunability of this approach. While not tested in this example, it is known that the hydrophobicity and presence of carbon units between an ester and a thiol can affect the rate of ester hydrolysis (Zustiak, S. P.; Leach, J. B. Hydrolytically Degradable Poly (Ethylene Glycol) Hydrogel Scaffolds with Tunable Degradation and Mechanical Properties. Biomacromolecules 2010, 11 (5), 1348-1357, Jo, Y. S.; Gantz, J.; Hubbell, J. A.; Lutolf, M. P. Tailoring Hydrogel Degradation and Drug Release via Neighboring Amino Acid Controlled Ester Hydrolysis. Soft Matter 2009, 5 (2), 440-446, and Schoenmakers, R. G.; van de Wetering, P.; Elbert, D. L.; Hubbell, J. A. The Effect of the Linker on the Hydrolysis Rate of Drug-Linked Ester Bonds. Journal of Controlled Release 2004, 95 (2), 291-300). Thus, the implementation of linkers with hydrophobic molecular units between the ester and the thiol group or alterations to the polymer density may provide further control over the degradation of hydrogels synthesized by this approach without any appreciable impact on the fabrication technique.

Material degradability, while highly desirable for biomaterial platforms, can lead to unwanted toxicity and immune activation responses that hamper their applicability in the clinic. In this example, no noticeable effects on cell viability were observed in any of the microgel formulations tested, demonstrating that the presence of microgels or their degradation byproducts do not result in toxicity-induced cell death at a dosage of up to approximately 3 microgels/μL. Additionally, the size of the microgels implemented in this example (>200 μm) should prevent them from being phagocytosed by macrophages (Champion, J. A.; Mitragotri, S. Role of Target Geometry in Phagocytosis. PNAS 2006, 103 (13), 4930-4934); however, degradation by-products and partial internalization leads to macrophage polarization. Although changes in mechanical cues have demonstrated an effect on MI-like macrophage activation (Patel, N. R.; Bole, M.; Chen, C.; Hardin, C. C.; Kho, A. T.; Mih, J.; Deng, L.; Butler, J.; Tschumperlin, D.; Fredberg, J. J.; Krishnan, R.; Koziel, H. Cell Elasticity Determines Macrophage Function. PLOS ONE 2012, 7 (9), e41024 and Féréol, S.; Fodil, R.; Labat, B.; Galiacy, S.; Laurent, V. M.; Louis, B.; Isabey, D.; Planus, E. Sensitivity of Alveolar Macrophages to Substrate Mechanical and Adhesive Properties. Cell Motility 2006, 63 (6), 321-340), changes in surface chemistry have been shown to have a higher influence on M2-like polarization (Thiols Decrease Human Interleukin (IL) 4 Production and IL-4-Induced Immunoglobulin Synthesis. J Exp Med 1995, 182 (6), 1785-1792 and Li, Z.; Bratlie, K. M. How Cross-Linking Mechanisms of Methacrylated Gellan Gum Hydrogels Alter Macrophage Phenotype. ACS Appl. Bio Mater. 2019, 2 (1), 217-225). In this example, neither the presence of EGBMA crosslinking units nor changes in mechanical properties had an impact on M2-associated CD206 marker expression in vitro. Most of the unreacted crosslinking molecules were removed from the microgel suspension through the centrifugation/washing steps. Furthermore, at the microgel to cell ratio implemented, the concentration of the linker molecules in solution, due to hydrolysis, did not influence this marker expression.

Despite several strategies reported for degradable PEG-based hydrogels, few studies have reported that in vivo degradation rates can be orders of magnitude different those in vitro (Hunckler, M. D.; Medina, J. D.; Coronel, M. M.; Weaver, J. D.; Stabler, C. L.; García, A. J. Linkage Groups within Thiol-Ene Photoclickable PEG Hydrogels Control In Vivo Stability. Advanced Healthcare Materials 2019, 8 (14), 1900371, Amer, L. D.; Bryant, S. J. The in Vitro and in Vivo Response to MMP-Sensitive Poly (Ethylene Glycol) Hydrogels. Ann Biomed Eng 2016, 44 (6), 1959-1969, and Browning, M. B.; Cereceres, S. N.; Luong, P. T.; Cosgriff-Hernandez, E. M. Determination of the in Vivo Degradation Mechanism of PEGDA Hydrogels. J Biomed Mater Res A 2014, 102 (12), 4244-4251). Indeed, protease-cleavable formulations that have been shown to rapidly degrade in culture do not degrade post-implantation (Amer, L. D., Bryant, S. J. The in Vitro and in Vivo Response to MMP-Sensitive Poly (Ethylene Glycol) Hydrogels. Ann Biomed Eng 2016, 44 (6), 1959-1969). Likewise, differences in degradation rates have also been observed in hydrolytic degradation, whereby the gradual hydrolytic degradation rates in vitro did not match the rapid degradation observed in vivo (Hunckler, M. D.; Medina, J. D.; Coronel, M. M.; Weaver, J. D.; Stabler, C. L.; García, A. J. Linkage Groups within Thiol-Ene Photoclickable PEG Hydrogels Control In Vivo Stability. Advanced Healthcare Materials 2019, 8 (14), 1900371). Here, using microgels labeled with a near-infrared dye, it was demonstrated that DTT/EGBMA-crosslinked microgels degrade in vivo, with degradation times that span several weeks. This is consistent with the degradation rates observed in the in vitro studies and to other ester-containing bulk PEG hydrogels (Zustiak, S. P.; Leach, J. B. Hydrolytically Degradable Poly (Ethylene Glycol) Hydrogel Scaffolds with Tunable Degradation and Mechanical Properties. Biomacromolecules 2010, 11 (5), 1348-1357). In subsequent studies, it will be important to evaluate how the addition of biological factors (e.g., adhesion ligands, encapsulated cells, or therapeutics) alters the rate of ester hydrolysis in these microgels.

Finally, tissue responses as a function of degradability were assessed in a subcutaneous dorsal model. This site provides an easily accessible location that can hold substantial microgel transplant volumes. Moreover, it permits the use of the same animal as its own internal positive control, as multiple independent microgel suspensions can be injected into different quadrants of the dorsum. Multiparametric flow analysis demonstrated degradation-dependent immune responses, with the enhanced presence of myeloid and T cells, in particular CD4+ cells, in the nondegradable formulation, consistent with other studies showing T helper cells driving responses to synthetic material implants (Chung, L.; Maestas, D. R.; Lebid, A.; Mageau, A.; Rosson, G. D.; Wu, X.; Wolf, M. T.; Tam, A. J.; Vanderzee, I.; Wang, X.; Andorko, J. I.; Zhang, H.; Narain, R.; Sadtler, K.; Fan, H.; Čiháková, D.; Le Saux, C. J.; Housseau, F.; Pardoll, D. M.; Elisseeff, J. H. Interleukin 17 and Senescent Cells Regulate the Foreign Body Response to Synthetic Material Implants in Mice and Humans. Sci Transl Med 2020, 12 (539), eaax3799 and Chan, T.; Pek, E. A.; Huth, K.; Ashkar, A. A. CD4+ T-Cells Are Important in Regulating Macrophage 20) Polarization in C57BL/6 Wild-Type Mice. Cellular Immunology 2011, 266 (2), 180-186). Further evaluation of the cytokine environment provided additional insights into the diversity and complexity of the immune responses. In contrast to reports of an IL-17-driven immune response to synthetic bulk implants (Chung, L.; Maestas, D. R.; Lebid, A.; Mageau, A.; Rosson, G. D.; Wu, X.; Wolf, M. T.; Tam, A. J.; Vanderzee, I.; Wang, X.; Andorko, J. I.; Zhang, H.; Narain, R.; Sadtler, K.; Fan, H.; Čiháková, D.; Le Saux, C. J.; Housseau, F.; Pardoll, D. M.; Elisseeff, J. H. Interleukin 17 and Senescent Cells Regulate the Foreign Body Response to Synthetic Material Implants in Mice and Humans. Sci Transl Med 2020, 12 (539), eaax3799), it was found that injections of synthetic microgel suspensions led to a prominent expression of IFN-γ which remained elevated for up to 4 weeks. Remarkably, unsupervised clustering of cytokine correlations identified IFN-γ as the dominant response driving cytokine communications. IFN-γ is one of the canonical cytokines driving type 1 immune responses (Tuzlak, S.; Dejean, A. S.; Iannacone, M.; Quintana, F. J.; Waisman, A.; Ginhoux, F.; Korn, T.; Becher, B. Repositioning TH Cell Polarization from Single Cytokines to Complex Help. Nat Immunol 2021, 22 (10), 1210-1217), and it is primarily produced by activated T cells and promotes MI polarization by STAT1 phosphorylation (Kak, G.; Raza, M.; Tiwari, B. K. Interferon-Gamma (IFN-γ): Exploring Its Implications in Infectious Diseases. Biomolecular Concepts 2018, 9 (1), 64-79). Evaluation of the macrophage cellular response acutely post-implantation of the microgel suspension revealed phenotypic characteristics that resemble more an M2-like phenotype (i.e., CD206 expression). This phenotypic plasticity demonstrates a shift in the microenvironment milieu leading to repolarization of IFN-γ-activated macrophages. Future work should investigate if indeed this repolarization is due to the presence of other cytokines in the immune response (i.e. persistence of IL-4), given that MI polarization can prime the transition into distinct M2 phenotypes in response to IL-4(O'Brien, E. M.; Spiller, K. L. Pro-Inflammatory Polarization Primes Macrophages to Transition into a Distinct M2-like Phenotype in Response to IL-4. Journal of Leukocyte Biology n/a (n/a)). Additionally, these studies were performed in BALB/cJ mice which have shown to have a genetic predisposition towards M2 polarization. Thus, a microgel-induced M2 phenotype cannot be generalized until tested in other strains (Chan, T.; Pek, E. A.; Huth, K.; Ashkar, A. A. CD4+ T-Cells Are Important in Regulating Macrophage Polarization in C57BL/6 Wild-Type Mice. Cellular Immunology 2011, 266 (2), 180-186).

Although type 1 cytokines seemed to be the primary driver of local tissue responses, this example shows a reciprocal IL-4-driven response that should be further investigated. No observable changes in IL-4 secretion even after 30 days post-injection were evident in the nondegradable implant. Notably, modulation of this immune response was possible by conferring a degree of degradability to the microgel platform, in that hydrolytically degradable microgels saw a decrease in IL-4 secretion and corresponding reduction in CD206+ macrophage presence compared to non-degradable microgels. Even minimal incorporation of the labile ester crosslinker, which does not lead to full degradation in the time window tested (i.e 0.25 mM EGBMA), provided differences in cellular and cytokine profiles, showing that the degradation profile of the material greatly influences the tissue response.

Although not investigated in this example, a range of parameters such as geometry, size, surface texture, stiffness and charge of materials can influence the host-implant interaction and the subsequent immune recognition and development of a FBR (Doloff, J. C.; Veiseh, O.; de Mezerville, R.; Sforza, M.; Perry, T. A.; Haupt, J.; Jamiel, M.; Chambers, C.; Nash, A.; Aghlara-Fotovat, S.; Stelzel, J. L.; Bauer, S. J.; Neshat, S. Y.; Hancock, J.; Romero, N. A.; Hidalgo, Y. E.; Leiva, I. M.; Munhoz, A. M.; Bayat, A.; Kinney, B. M.; Hodges, H. C.; Miranda, R. N.; Clemens, M. W.; Langer, R. The Surface Topography of Silicone Breast Implants Mediates the Foreign Body Response in Mice, Rabbits and Humans. Nat Biomed Eng 2021, 5 (10), 1115-1130, Veiseh, O.; Doloff, J. C.; Ma, M.; Vegas, A. J.; Tam, H. H.; Bader, A. R.; Li, J.; Langan, E.; Wyckoff, J.; Loo, W. S.; Jhunjhunwala, S.; Chiu, A.; Siebert, S.; Tang, K.; Hollister-Lock, J.; Aresta-Dasilva, S.; Bochenek, M.; Mendoza-Elias, J.; Wang, Y.; Qi, M.; Lavin, D. M.; Chen, M.; Dholakia, N.; Thakrar, R.; Lacik, I.; Weir, G. C.; Oberholzer, J.; Greiner, D. L.; Langer, R.; Anderson, D. G. Size- and Shape-Dependent Foreign Body Immune Response to Materials Implanted in Rodents and Non-Human Primates. Nature Mater 2015, 14 (6), 643-651, and Blakney, A. K.; Swartzlander, M. D.; Bryant, S. J. The Effects of Substrate Stiffness on the in Vitro Activation of Macrophages and in Vivo Host Response to Poly (Ethylene Glycol)-Based Hydrogels. J Biomed Mater Res A 2012, 100 (6), 1375-1386). For example, FBR to spherical agarose microgels is modulated by the geometry and size of the implant, with larger sphere implants activating a lower FBR compared to smaller implants (Veiseh, O.; Doloff, J. C.; Ma, M.; Vegas, A. J.; Tam, H. H.; Bader, A. R.; Li, J.; Langan, E.; Wyckoff, J.; Loo, W. S.; Jhunjhunwala, S.; Chiu, A.; Siebert, S.; Tang, K.; Hollister-Lock, J.; Aresta-Dasilva, S.; Bochenek, M.; Mendoza-Elias, J.; Wang, Y.; Qi, M.; Lavin, D. M.; Chen, M.; Dholakia, N.; Thakrar, R.; Lacik, I.; Weir, G. C.; Oberholzer, J.; Greiner, D. L.; Langer, R.; Anderson, D. G. Size- and Shape-Dependent Foreign Body Immune Response to Materials Implanted in Rodents and Non-Human Primates. Nature Mater 2015, 14 (6), 643-651). Likewise, chemical modification of PEG hydrogels with hydrophilic materials can modulate the FBR by reducing protein absorption and cellular attachment (Jansen, L. E.; Amer, L. D.; Chen, E. Y.-T.; Nguyen, T. V.; Saleh, L. S.; Emrick, T.; Liu, W. F.; Bryant, S. J.; Peyton, S. R. Zwitterionic PEG-PC Hydrogels Modulate the Foreign Body Response in a Modulus-Dependent Manner. Biomacromolecules 2018, 19 (7), 2880-2888). Important material properties such as stiffness are increasingly recognized to have a profound impact on driving cellular behaviors (Blakney, A. K.; Swartzlander, M. D.; Bryant, S. J. The Effects of Substrate Stiffness on the in Vitro Activation of Macrophages and in Vivo Host Response to Poly (Ethylene Glycol)-Based Hydrogels. J Biomed Mater Res A 2012, 100 (6), 1375-1386 and Irwin, E. F.; Saha, K.; Rosenbluth, M.; Gamble, L. J.; Castner, D. G.; Healy, K. E. Modulus-Dependent Macrophage Adhesion and Behavior. Journal of Biomaterials Science, Polymer Edition 2008, 19 (10), 1363-1382). Stiffness-driven inflammatory responses to PEG hydrogels have been previously reported and thought to be associated to an increased immune cellular adhesion to stiffer surfaces (Blakney, A. K.; Swartzlander, M. D.; Bryant, S. J. The Effects of Substrate Stiffness on the in Vitro Activation of Macrophages and in Vivo Host Response to Poly (Ethylene Glycol)-Based Hydrogels. J Biomed Mater Res A 2012, 100 (6), 1375-1386). This response has been recently attributed to the mechanosensitive transient receptor potential vanilloid 4 (TRPV4) independently of other biochemical cues (Goswami, R.; Arya, R. K.; Sharma, S.; Dutta, B.; Stamov, D. R.; Zhu, X.; Rahaman, S. O. Mechanosensing by TRPV4 Mediates Stiffness-Induced Foreign Body Response and Giant Cell Formation. Science Signaling 14 (707), eabd4077). The microgel implants used in this work swell significantly during hydrolysis-mediated degradation, and thus it cannot be ruled out that this dynamic shift in size post-implantation, together with decreasing stiffness leads to the modulation of cell infiltration observed in this example. It would be of interest to decouple these two parameters in the nondegradable implants to evaluate the singular effect of size of DTT crosslinked microgels on the FBR.

In sum, this example presents a cost-effective approach to conferring microgels with degradable features from PEG-4MAL macromers segmented via droplet microfluidics. Microgels with ester labile crosslinking junctions readily degrade in vitro and in vivo. Furthermore, the degradation profile impacts the immune response to the implant, with reduced type 1 associated cytokines and cells present when degradable microgels are delivered. The simplicity of this strategy and the efficiency of hydrolytic degradation of the resulting microgel population makes this approach attractive for regenerative medicine and drug delivery applications.

### Experimental

Microfluidic Device Fabrication: PDMS microfluidic devices were prepared as previously reported (Headen, D. M.; Aubry, G.; Lu, H.; García, A. J. Microfluidic-Based Generation of Size-Controlled, Biofunctionalized Synthetic Polymer Microgels for Cell Encapsulation. Advanced Materials 2014, 26 (19), 3003-3008). In brief, PDMS was cast using soft lithography and SU8 masters with microfluidic device patterns and heated to 110 ºC for 20 minutes. The resulting PDMS microfluidic devices were removed from the wafer and bonded to glass slides and heated overnight to 70° C.

PEG-4MAL Microgel Fabrication: Polymer droplets were formed using a flow focusing microfluidic device with a 200 μm nozzle. The aqueous phase consisted of a 5% w/v PEG-4Mal (20 KDa, Laysan Bio) which had been previously reacted with a thiol-PEG-FITC (1 kDa, Nanocs). A co-flowing shielding phase consisted of mineral oil (Sigma) with 2% SPAN80 (Sigma). The crosslinker phase contained an emulsion of mineral oil/SPAN80 with DTT (Thermo) at a concentration of 15 mM. To render the microgels degradable various amounts of EGBMA (Sigma) were added to this crosslinker phase at 0.25, 0.5 and 1.0 mM concentrations. After fabrication, microgels were extracted from the oil phase by centrifugation, and washed with a 2% bovine serum albumin (Sigma)/PBS (corning) solution.

Microgel Sizing and Swelling: Characterization of crosslinking phase on microgel size was measured after fabrication using a Biotek Cytation spectrophotometer. A sample of 50 μL in triplicates was placed in a glass bottom 6-well plate. Quantitative fluorescent intensity for each microgel was recorded for all samples. Droplet diameter was measured using the cellular analysis plug-in in the Cytation Gen software. For swelling studies, 1000 microgels were placed in 1 mL of PBS and placed in the incubator. Samples of 50 μL were taken every day and measured as described above. For FITC tracking studies, 1000 microgels were placed in 1 mL of PBS and solution was replaced every day. Collected supernatant fluorescence was measured using a Cytation 3 plate reader.

Microcapillary Mechanical Testing: Microgel elastic properties were determined using pressure-driven capillary micromechanics (Wyss, H. M.; Franke, T.; Mele, E.; Weitz, D. A. Capillary Micromechanics: Measuring the Elasticity of Microscopic Soft Objects. Soft Matter 2010, 6 (18), 4550-4555). At various time points (day 0, 3, 7), a microgel was inserted into the end of a tapered glass micropipette (Fivephoton Biochemicals) precoated with 1% (w/v) BSA in PBS. A high precision pressure regulator (Elveflow) was attached to the end of the micropipette, and pressure applied at various intervals (0, 2.5, 5, 7.5, 10, 15, 20, 25, 30, 40, 50, 60 kPa). When the microgel reached equilibrium (no longer moving in micropipette when external applied pressure balanced with internal elastic stress), an image was acquired on a microscope (10×; EVOS), and parameters were measured using ImageJ.

Viability assessments: RAW 264.7 cells were co-cultured with 10,000 microgels for 7 days. Cell metabolic activity was measured via AlamarBlue (Invitrogen). The assay was performed at different time points (1, 2, 4 and 7 days). After 4 h of incubation, 100 μL of the supernatant was transferred to the wells of a 96-well plate and the OD was measured using a Cytation 3 imaging reader (Biotek) at 570 nm and 600 nm wavelengths.

Bone Marrow Derived Macrophage Co-culture: Bone marrow was isolated from the femurs and tibias of 6-week-old male C57BL/6J mice. Bones were cleaned of soft tissue, one side was cut to expose the marrow, and they were inverted in a 200 μL pipet tip cut to fit in a 1.5 mL Eppendorf tube. The bones were then centrifuged at 10,000×g for 15 sec to pellet the marrow in the bottom of the Eppendorf tube. Bones were discarded and cells were then resuspended in RBC Lysis Buffer (Biolegend 420302) to remove red blood cells. Cells were then washed in MACS buffer (DPBS pH 7.2, 0.5% BSA, 2 mM EDTA) and monocytes were isolated using the Monocyte Isolation Kit (BM), mouse (Miltenyi Biotec 130-100-629) and LS columns (Miltenyi Biotec 130-042-401).

Monocytes were cultured in RPMI 1640 media (Gibco 11875-085) supplemented with 10% heat-inactivated fetal bovine serum, 1% pen/strep, and 20 ng/mL murine M-CSF (Biolegend 574804) for 6 days in low-adherent plates. Cells were harvested and seeded with microparticles at a 1:10 ratio (10,000 cells/1000 microgels per well). M2 control macrophages were cultured in media supplemented with both 20 ng/ml murine M-CSF and 20 ng/ml murine IL-4 (Biolegend 574304). After 48 and 96 hr of co-culture cells were harvested and stained using the following markers: live dead (Zombie Violet, BioLegend 423113), CD45 (PE-Texas Red, BioLegend 103146), CD11b (PercpCy5.5, BioLegend 101228), F4/80 (FITC, BioLegend 123108), and CD206 (PECy7, BioLegend 141720). Samples were analyzed on a FACS-AriaIIIu flow cytometer (BD Biosciences).

Transplantation of microgels into mice: All animal procedures were performed under protocols approved by Georgia Institute of Technology IACUC and in accordance with National Institutes of Health guidelines (IACUC approved protocol number A100326). Microgels were injected under the epidermis of 8-12-week-old BALB/cJ mice. The 100 μL injections consisted of about 3000 nondegradable or degradable hydrogels. All four conditions were injected into the same animal at independent sites to reduce any variability due to inherent biological differences across animals.

Microgel In Vivo Tracking: Macromer was functionalized with a 1 KDa PEG labelled with AlexaFluor750 NHS ester (Thermo Fisher). Immediately after fabrication, 3000 microgels were injected under the epidermis in 100 μL of saline. Signal intensity and distribution were monitored longitudinally using an IVIS SpectrumCT imaging system (Perkin-Elmer). Data was analyzed using Living Image software. Regions of interest (ROIs) were drawn in defined pocket areas and quantified using Radiant Efficiency [p/s/sr]/[μW/cm2]. The ROIs were kept the same size for each group pocket at all time points and were appropriately sized to contain the fluorescent signal for each region, to ensure that the imaging data between individual donors can be compared across time. Intensity measurements were normalized to day 0 values.

MultiParametric Flow Analysis of Tissue Responses: Tissue samples were obtained by a 12 mm biopsy punch and digested for 60 min at 37ºC with an Accumax solution (Sigma). The digested tissue was passed through a 40 μm strainer and then washed twice with 1×PBS. Cells were washed stained for live/dead (Zombie violet, BioLegend 423113) and surface-stained with myeloid markers: CD45 (PE-Texas Red, BioLegend 103146), CD11b (PercpCy5.5, BioLegend 101228), F4/80 (FITC, BioLegend 123108), CD11c (BV785, BioLegend 117335), MHCII (APC-Cy7, BioLegend 107652), CD86 (APC, BioLegend 105012), CD206 (PECy7, BioLegend 141720). As well as lymphoid markers: CD45 (BV711, BioLegend), CD3 (BV510, BioLegend 100233), CD4 (APC, BioLegend 100412), CD8 (PercpCy5.5, BioLegend 100732), CD25 (PECy7, BioLegend 102016), PD-1 (PE Texas Red, BioLegend 135227). Flow cytometry was performed with an BD Aria and analyzed in FCS express.

Cytokine Analysis: Microgels were injected subcutaneously under the epidermis as described above. At set time points, a 12 mm biopsy punch in the surrounding injection site was used to remove the tissue. Samples were subsequently placed in RIPA buffer containing a protease inhibitor (Thermo). Samples were sonicated and centrifuged at 10,000×g for 10 min at 4° ° C. to remove debris. Supernatant was frozen in liquid nitrogen and stored at −80° C. until analysis. Samples were analyzed using the Milliplex MAP Mouse Cytokine/Chemokine 32-plex assay (Millipore, MCYTMAG) on a Magpix multiplexing machine (Luminex) according to the manufacturer's instructions. PCA was conducted on all samples using the “prcomp” function in R and visualized using the “factoextra” package. Cytokine correlations were investigated using CytoMod (Cohen, L.; Fiore-Gartland, A.; Randolph, A. G.; Panoskaltsis-Mortari, A.; Wong, S.-S.; Ralston, J.; Wood, T.; Seeds, R.; Huang, Q. S.; Webby, R. J.; Thomas, P. G.; Hertz, T. A Modular Cytokine Analysis Method Reveals Novel Associations With Clinical Phenotypes and Identifies Sets of Co-Signaling Cytokines Across Influenza Natural Infection Cohorts and Healthy Controls. Frontiers in Immunology 2019, 10, 1338 and Distinct inflammatory profiles distinguish COVID-19 from influenza with limited contributions from cytokine storm https://www.science.org/doi/10.1126/sciadv.abe3024 (accessed 2021-10-25)). For correlation analysis, values below the lower limit of detection were set to the lower limit of detection. Multi-variate linear regression with Log₁₀ concentrations were modeled as a function of time. The “emmeans” package in R was used to assess pairwise differences in estimated marginal means between conditions, and Tukey's method was used to adjust for multiple comparisons.

Immunohistochemistry: After euthanasia at day 30, a 12 mm biopsy punch in the surrounding injection site was used to remove the tissue, which was then fixed in 10% formalin solution overnight. Samples were subsequently processed with dehydration in graded ethanol solutions, cleared in xylene and paraffin-embedded. Sections were cut at 10 μm and slides were stained using hematoxylin and eosin (H&E), and IHC for macrophage pan marker CD68 (abcam, ab125212), and a nuclear staining (DAPI, Invitrogen D1306).

Statistical Analysis: All experiments were performed on biological replicates. Sample size for each experimental group and statistical test used, with post hoc test where appropriate, to determine significant differences among groups are reported in the appropriate figure legend. Exact p values or meaning of significance symbol are presented in the legend. Data was analyzed with Graphpad Prism v9 (GraphPad Inc.). For cytokine analysis, concentration data was log transformed for normalization; analysis was performed in R, using the hclust, glm and lmmeans package. Experiments were not blinded, and no randomization was used.

### Example 2. Degradable Microgels

Hydrogel crosslinking with ester containing linkers offers a degradation mechanism focused on hydrolytic cleavage of the ester bond. Degradation can be controlled by polymer content, molecular weight, and crosslinking density of the ester labile linker (Zustiak, S. P., & Leach, J. B. (2010). Hydrolytically Degradable Poly(Ethylene Glycol) Hydrogel Scaffolds with Tunable Degradation and Mechanical Properties. Biomacromolecules, 11(5), 1348-1357). Contrasting from enzymatic-dependent hydrogels, the hydrogels developed through this approach are degradable through hydrolysis allowing for a controlled, consistent degradation profile dependent solely on the adjustable physical, mechanical, and chemical properties of the hydrogel (Sung, B., Kim, C., & Kim, M.-H. (2015). Biodegradable colloidal microgels with tunable thermosensitive volume phase transitions for controllable drug delivery. Journal of Colloid and Interface Science, 450, 26-33 and Stukel, J., Thompson, S., Simon, L., & Willits, R. (2015). Polyethlyene glycol microgels to deliver bioactive nerve growth factor: Microgels to Deliver Bioactive NGF. Journal of Biomedical Materials Research Part A, 103(2), 604-613). Importantly, this method of degradation does not obstruct the implementation of cysteine-terminated adhesives or peptides (Stukel, J., Thompson, S., Simon, L., & Willits, R. (2015). Polyethlyene glycol microgels to deliver bioactive nerve growth factor: Microgels to Deliver Bioactive NGF. Journal of Biomedical Materials Research Part A, 103(2), 604-613). Its uses in drug delivery and tissue engineering have proven the method to be effective in offering a long-term, sustained release profile (Zustiak, S. P., & Leach, J. B. (2010). Hydrolytically Degradable Poly(Ethylene Glycol) Hydrogel Scaffolds with Tunable Degradation and Mechanical Properties. Biomacromolecules, 11(5), 1348-1357, Pradal, C., Grøndahl, L., & Cooper-White, J. J. (2015). Hydrolytically Degradable Polyrotaxane Hydrogels for Drug and Cell Delivery Applications. Biomacromolecules, 16(1), 389-403, and Davis, K. A., & Anseth, K. S. (2002). Controlled Release from Crosslinked Degradable Networks; Critical Reviews in Therapeutic Drug Carrier Systems, 19(4-5), 385-424). Herein, we present a fabrication mechanism that produces monodisperse hydrolytically degradable hydrogels, based on flow-focusing droplet generation, with tunable release and degradation profiles dependent on the introduction of a labile ethylene linker. Tuning the labile ester chemistry of the hydrogel structure via the addition of varying amounts of ethylene linker and a nondegradable thiol linker, controlled hydrogel degradation profiles can be developed. Additionally, we characterize the effects of changes in mechanical properties, and degradable byproducts on cell phenotypes in vitro and in vivo.

### Methods

Microfluidic device fabrication. PDMS microfluidic devices were constructed from the addition of 184 silicone elastomer and 184 silicon elastomer curing agent. The silicone mixture was then placed on a silicon wafer consisting of microfluidic device patterns and heated to 110° C. for 20 minutes. The resulting PDMS microfluidic devices were removed from the wafer and bonded to glass slides and heated overnight to 70° C.

4-arm poly (ethylene glycol) microgel construction. PEG-4Mal (20 KDa four-armed polyethylene glycol from Laysan Bio), PEG biotin, and DTT (Dithiothreitol) were weighed to the appropriate amounts. A10 mM DPBS/HEPES (Dulbecco's phosphate-buffered saline/4-(2-hydroxyethyl)-1-piperazineethanesulfonic acid) solution was made which was used to suspend the PEG biotin in. This new solution consisting of the DPBS/HEPES solution and Peg biotin was then used to resuspend the PEG-4Mal, which flowed through a line to the microfluidic device. Additionally, a DTT and DPBS/HEPES solution was made. A 2% SPAN 80/mineral oil solution was also made and 395 ul of DTT in DPBS/HEPES was added to 5 mL of the 2% SPAN 80/mineral oil solution. For degradable microgels, a calculated concentration of degradable thiol linker was added to the solution of DTT and 2% SPAN 80/mineral oil. Following the creation of the solutions, three syringe pumps were set up and the microfluidic device was primed for fabrication. One pump was set to push oil through the device (1 ul/min), another pump was set to push the PEG through the device (5 ul/min), and the final pump was used to push the DTT solution through the device (35 ul/min). A collection line was set up from the collection bath in the device to a collection tube filled with dPBS and 1% BSA (Bovine Serum Albumin). After priming the device, the lines were set up and the three solutions were run through the microfluidic device. After running the pumps and lines through the device for approximately 45 minutes, the collection tube was placed in the centrifuge for five minutes, and a series of washes was done to remove the DTT and oil from the collection resulting in a collection of microgels at the bottom of the tube.

Microgel degradation. Approximately 200 microgels were placed into each well of a 48-well plate and incubated over the course of multiple days. Each day, the number of microgels in each well was counted and analyzed for swelling using a LED microscope. After analyzing the microgels, DPBS was added to each well and placed back into the incubator.

Semi-quantitate analysis of protein encapsulation. A Western Blot Transfer was conducted to analyze the ability of the hydrogels to capture proteins. Through the techniques of gel electrophoresis and chemiluminescence imaging, a semi-quantitative analysis of the microgels was conducted to determine whether the microgels were ready for transplant.

Transplantation of microgels into mice. Microgels were injected under the epidermis of 8-12 week old Balb/C mice. The 100 μL injections consisted of about 3000 nondegradable or degradable hydrogels. The nondegradable microgels consisted of DTT crosslinker and no degradable thiol linker while the degradable microgels consisted of a mixture of DTT crosslinker and 0.25 mM, 0.5 mM, or 1 mM thiol linker.

Microgel tracking. Using an IVIS imaging system, microgel imaging and its fluorescence could be seen. Over the course of several days, the microgel signaling was tracked and recorded for degradation rates.

### Example 3. Hydrolytically Degradable Hydrogels for Therapeutic Delivery

Hydrogels are increasingly used in regenerative medicine for the delivery of drugs or biological therapeutic agents, as they are modular, biocompatible, and can be engineered to have controllable mechanical properties. Increasingly, degradable hydrogels are fabricated using sequence-specific enzymatic degradation of peptides incorporated into hydrogels. Whereas this degradation method is cytocompatible, the poor solubility of the peptides in oil, and the requirements for high volume of peptide solution limits the synthesis of monodisperse degradable hydrogels with microfluidic devices. Here, hydrolytically degradable hydrogels with tunable degradations are reported based on labile chemistry responsive to endogenous stimuli (i.e. hydrolysis).

### Methods

Hydrogel particles (microgels) were fabricated in a microfluidic water-in-oil droplet generator as previously described (Headen et al. Microsystems & Nanoengineering 4.1 (2018): 1-9). Polymer was prefunctionalized with a 1 kDa SH-PEG-FITC for in vitro tracking or a SH-PEG-AF750 for in vivo imaging. Microgels were crosslinked with a solution containing: dithiothreitol (DTT), or a mixture of DTT and a degradable linker ethylene glycol bis-mercaptoacetate at different molar ratios. Microgels were injected under the skin of mice for in vivo tracking. Briefly, 8-12 week old Balb/C mice were injected with 100 μL of ˜3000 nondegradable hydrogels (DTT crosslinked) or degradable hydrogels (mix of DTT and 0.25 mM, 0.5 mM, or 1 mM degradable linker).

### Results

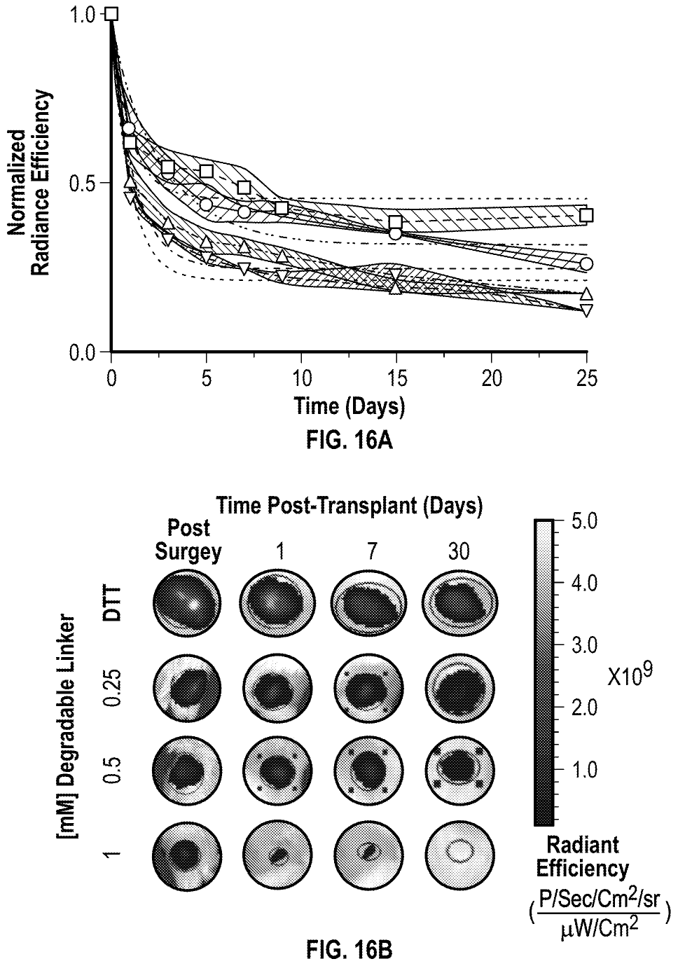

Degradation of microgels was tracked by measuring the swelling percentage and presence of PEG-FITC linker in solution of microgels culture at 37° ° C. in dPBS. Swelling percentage was found to be related to the molar concentration of degradable linker upon fabrication (FIG. 1F). Highly degradable hydrogels swelled ˜40% when compared to nondegradable hydrogels (p<0.0001) within 4 hours post-fabrication. By a month post culture, 1 mM degradable hydrogels had swelled to 60% of the size of the DTT nondegradable hydrogels (P<0.0039). No differences in size were observed between the DTT, 0.25 mM and 0.5 mM group initially, yet by day 30 they had increased size by 30% when compared to nondegradable gels (p<0.0001, p<0.019 respectively). Degradation of the hydrogels will lead to cleavage of the PEG-FITC linker from the PEG-4MAL backbone (FIG. 1B). As such, the fluorescence intensity of the culture was tracked overtime to determine the presence of PEG-FITC in solution. As observed in the swelling studies, the higher fluorescence intensity was in the 1 mM degradable hydrogel group (p<0.001 vs DTT). While no significant differences were observed when comparing the lowest degradable gel with the DTT crosslinked hydrogels. To evaluate in vivo degradation, microgels were monitored using an IVIS imaging system (FIGS. 16A-B). Immediately post-injection a strong signal was detected in all groups. By day 1 signal had decayed by about 38% in all groups, which can be attributed to the presence of free dye during fabrication, and not to degradation. By day 3, signal had decreased to 33% in the 1 mM hydrogel group, while a constant signal remained at the DTT injection site. By Day 10, no detectable signal was observed in the highly degradable group, whereas no changes were observed in the DTT microgels group.

### CONCLUSION

Monodisperse hydrolytically degradable hydrogels can be prepared implementing microfluidic water-in-oil droplet generators. The implementation of an ethylene linker together with a nondegradable thiol linker allows for a controllable sustained material degradation in vitro and in vivo.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

The term “comprising”, and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A hydrogel comprising a polymer backbone crosslinked with a first crosslinker containing at least one a moiety of Formula I: 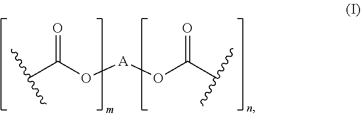
wherein:
m and n are independently 1 or 2;
A is C₂-C₁₀ alkyl; and 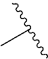
is a point of attachment for the moiety within the first crosslinker.

2-7. (canceled)

8. The hydrogel of claim 1, wherein the polymer backbone comprises a poly(ethylene glycol) or a functionalized derivative thereof.

9. The hydrogel of claim 1, wherein the polymer backbone comprises a polymer selected from poly(ethylene glycol) (PEG), poly(ethylene glycol)-di-acrylate (PEG-DA), multi-arm poly(ethylene glycol)-acrylate (PEG-Ac), poly(ethylene glycol)-dithiol (PEG-diSH), poly(ethylene glycol)divinyl sulfone (PEG-diVS), multi-arm poly(ethylene glycol)vinyl sulfone (PEG-VS), poly(ethylene glycol)-di-methacrylate (PEG-DMA), multi-arm poly(ethylene glycol)-methacrylate (PEG-Mac), poly(ethylene glycol)-di-allyl ether (PEG-diAE), multi-arm poly(ethylene glycol)-allyl ether (PE-AD), poly(ethylene glycol)-di-vinyl ether (PEG-diVE), multi-arm poly(ethylene glycol)-vinyl ether (PEG-VE), poly(ethylene glycol)-di-maleimide (PEG-diMI), multi-arm poly(ethylene glycol)-maleimide (PEG-MI), poly(ethylene glycol)-di-norborene, multi-arm poly(ethylene glycol)norborene, poly(ethylene glycol-vinyl carbonate, multi-arm poly(ethylene glycol)-vinyl carbonate, and polyethylene glycol oligofumarate, or combinations thereof.

10. The hydrogel of claim 1, wherein the polymer backbone comprises multi-arm poly(ethylene glycol)-maleimide.

11. The hydrogel of claim 1, wherein the first crosslinker comprises m+n moieties capable of reacting with the polymer backbone, wherein m and n are as defined in claim 1.

12. The hydrogel of claim 1, wherein the first crosslinker comprises a compound of Formula II: 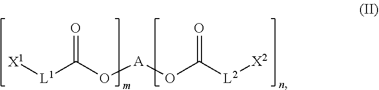
wherein:
X¹ and X² are independently selected at each occurrence from a moiety capable of reacting with the polymer backbone;
L¹ and L² are independently selected at each occurrence from a linking moiety; and
m, n, and A are defined as in claim 1.

13. The hydrogel of claim 12, wherein X¹ and X² are each —SH, and L¹ and L² are independently selected at each occurrence from C₁-C₁₀ alkyl.

14. (canceled)

15. The hydrogel of claim 1, wherein the first crosslinker comprises ethylene glycol bis(mercaptoacetate).

16. The hydrogel of claim 1, wherein the first crosslinker is hydrolytically degradable.

17. The hydrogel of claim 1, wherein the polymer backbone is further crosslinked with a second crosslinker.

18. The hydrogel of claim 17, wherein the second crosslinker is hydrolytically stable.

19. The hydrogel of claim 17, wherein the second crosslinker comprises dithiothreitol (DTT).

20. (canceled)

21. The hydrogel of claim 1, wherein the hydrogel is injectable and/or implantable.

22. The hydrogel of claim 1, wherein the hydrogel is in the form of a membrane, sponge, gel, solid scaffold, spun fiber, woven or unwoven mesh, nanoparticle, or microparticle.

23. The hydrogel of claim 1, further comprising at least one cell.

24. A process for synthesizing a hydrogel of claim 1 comprising reacting a polymer with a first crosslinker comprising at least one moiety of Formula I: 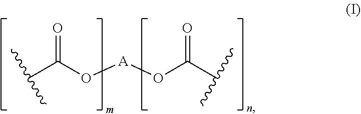
wherein all variables are as defined in claim 1.

25-28. (canceled)

29. A therapeutic delivery composition comprising a hydrogel of claim and one or more therapeutic agents.

30. The therapeutic delivery composition of claim 29, wherein the one or more therapeutic agents may be selected from a cell, a protein, an antibody, a nucleic acid, a growth factor, or a drug.

31-34. (canceled)

35. A method of promoting tissue growth in a subject in need thereof, comprising:
identifying a target site; and
administering a therapeutically effective amount of a hydrogel of claim 1 to the target site.

36-39. (canceled)

40. A method of delivering a therapeutic agent to a target site in a subject, the method comprising administering a therapeutically effective amount of a therapeutic delivery composition of claim 29 to the target site.

41-45. (canceled)
